(19) United States
(12) Patent Application Publication
(10) Pub. No.: US 2024/0383779 A1
(43) Pub. Date: Nov. 21, 2024

(54) Method for Controlling Radiation from a Source

(71) Applicant: 12180235 Canada Ltd., CentrePort (CA)

(72) Inventors: David Allan Prystupa, Pinawa (CA); John Stephen Pacak, Winnipeg (CA)

(21) Appl. No.: 18/692,898

(22) PCT Filed: Sep. 21, 2022
(86) PCT No.: PCT/CA2022/051397

§ 371 Date: Mar. 18, 2024

Related U.S. Application Data
(60) Provisional application No. 63/246,523, filed on Sep. 21, 2021.

(51) Int. Cl. C02F 1/32; A61L 2/10; A61L 2/26

(52) U.S. Cl. CPC C02F 1/325; A61L 2/10; A61L 2/26; A61L 2202/11; A61L 2202/122; C02F 2201/3228; C02F 2303/04

(57) ABSTRACT

Electromagnetic radiation is applied to reactive materials in a reaction chamber including side by side volumes where the probability of interaction of the electromagnetic radiation with the reactant materials is increased by using multiple reflections and where the reaction chamber includes a plurality of pairs of opposed reflective surfaces. At least 50% and more preferably at least 80% or 90% of the reflections from the reflective surfaces are specular reflections and one or both of the reflective surfaces of each pair is a concave mirror. The pairs are arranged side by side so that radiation escaping through a side of one volume enters a side of a next adjacent volume.

### RELATED APPLICATIONS

This disclosure is related to disclosures relating to a spectrometer disclosed in the patent entitled High Efficiency Multiplexing, hereafter “HEMS patent” by the present inventors described in U.S. Pat. No. 10,585,044 issued Mar. 10, 2020, the disclosures of which are incorporated herein by reference.

This disclosure is related to disclosures relating to a Multipass Photochemistry System, hereafter “MPS patent” by the present inventors described in U.S. patent applications Ser. Nos. 17/378,144, 17/378,154, 17/378,158, 17/378,163, 17/378,171, 17/378,175, and 17/378,186 all filed Jul. 16, 2021 the subject matter of which is published in in PCT Application PCT/CA2021/050976 filed Jul. 15 2021 and published as WO 2022/022472 on 20 Jan. 2022, the disclosures of which are incorporated herein by reference.

The invention relates to a method for controlling or directing radiation from a source and provides a number of novel aspects.

### BACKGROUND OF THE INVENTION

In many applications it is desirable maximize the radiation power that is passed through an aperture. In spectroscopic applications, the signal-to-noise ratio is proportional to the square root of the radiation power passed through a limiting aperture with less than a threshold angular divergence. In sterilization and photo-reactive applications, the sterilization rate or reaction rate is proportional to the radiation power passed through a limiting aperture with less than a threshold angular divergence. Hence it is desirable to have a method that increases the flux intensity passed through a limiting aperture.

The radiated power of a light source, whether emitted, scattered or reflected is proportional to its surface area and hence it is desirable to maximize the surface area from which radiated power can be passed through a limiting aperture. It is well established in the art to place a light source at a position centered at the focal point of an optical assembly and to image the light source onto and through an aperture. The aperture in turn may become the source for a second optical assembly that collimates radiation passed through the aperture. There are three major limitations to the amount of radiated power that can be passed through an aperture in prior art. The first limitation is the solid angle of the radiation source accepted by the optical assembly. That is power radiated in some directions is not collected by the optical assembly. The second limitation is that some of the radiation collected by the optical assembly is not imaged on and directed through the aperture. The third limitation is that a portion of radiation collected by the optical assembly is absorbed. The absorption consists of two parts: radiation absorbed by optical elements in the optical assembly and radiation directed by optical elements into the radiation source where absorption occurs. For example, in parabolic reflector systems of prior art, the radiation source is centered at the focal point (or plane) of a parabolic reflector and radiation emitted toward the vertex of the parabola is reflected back toward the focal point where it is absorbed with high efficiency. If the radiation source is a gas discharge lamp for example, a first radiation flux is emitted by atoms transitioning from an excited state to the ground state (or a less excited state). Radiation flux reflected back toward the focal point may in turn excite another atom of the same type from the ground state to an excited state. For a system in thermodynamic equilibrium, the ground state population is larger than the excited state population and absorption is consequently more probable than emission. The energy of an absorbed photon may be re-radiated as a second photon or the photon energy may be transferred to heat for example by atomic collisions. That is more energy is absorbed than is re-radiated.

### SUMMARY OF THE INVENTION

The arrangements described herein provide a number of aspects of the invention which are set out as follows.

Within the specification below the term “radiant body” refers to a region of space that emits a greater flux of radiation than it absorbs for at least one design wavelength.

Within the specification below the term and “radiation source” refers to a region of space that either emits or directs a net flux of radiation for at least one design wavelength. That is a radiation source may be a radiant body or an optical element such as a lens or mirror that collects and directs radiation from a radiant body.

Within the specification below, the term “radiation” from a “radiant body” or a “radiation source” refers to radiation at a design wavelength or within a range of design wavelengths. The methods described herein are generally applicable for radiation with wavelengths ranging from 200 nm to 100,000 nm, but the range of design wavelengths is generally much narrower. For sterilization applications, the design wavelength(s) may for example be between 220 nm and 280 nm. For lighting and illumination applications, the design wavelength(s) may be in the range between 400 nm and 750 nm. For near infrared spectroscopic measurements, the design wavelengths may for example be in the range from 800 nm to 2500 nm.

Within the specification below, the term “specular reflection” refers to reflections for which the difference between the angle of incidence and the angle of reflection is less than two degrees. That is the definition of specular reflection is extended from the ideal case of angle of reflection equals angle of incidence to include a narrow distribution of near specular angles. Radiation reflected (or scattered) at angles outside the specular range is defined as diffuse.

Within the specification below, the term “reflection” means specular reflection unless explicitly defined as diffuse.

Within the specification below, the term “mirror” means a smooth surface that reflects specularly.

According to one aspect of the invention which can be used independently or in combination with any of the other features described herein there is provided a method for collecting radiation emitted from a three-dimensional radiant body comprising:
collecting radiation emitted by the radiant body with a plurality of optical directing components;
wherein each optical directing component subtends less or equal to one half of the solid angle radiation is emitted into by said three-dimensional radiant body.

In accordance with one optional feature which can be used with the above definition or with other features defined herein, the radiant body has an axis of symmetry and the optical directing components are arranged symmetrically about the axis.

In accordance with one optional feature which can be used with the above definition or with other features defined herein, the collected radiation is directed to at least one aperture where an area of the aperture is less than a surface area of the three-dimensional radiant body.

In accordance with one optional feature which can be used with the above definition or with other features defined herein, the aperture consists of a plurality of apertures and the area of the apertures combined is less than the surface area of the radiation source.

In accordance with one optional feature which can be used with the above definition or with other features defined herein, each optical directing component subtends less or equal to one third of the solid angle radiation is emitted into by said three-dimensional radiant body

In accordance with one optional feature which can be used with the above definition or with other features defined herein, each optical directing component subtends less or equal to ⅙ of the solid angle radiation is emitted into by said three-dimensional radiant body.

In accordance with one optional feature which can be used with the above definition or with other features defined herein, there are six equiangular optical directing components around the radiant body.

In accordance with one optional feature which can be used with the above definition or with other features defined herein, the optical directing components comprise wave guides.

In accordance with one optional feature which can be used with the above definition or with other features defined herein, the radiation from each wave guide is directed into a radiation transfer element.

In accordance with one optional feature which can be used with the above definition or with other features defined herein, the optical directing components comprise lenses.

In accordance with one optional feature which can be used with the above definition or with other features defined herein, the collected radiation is greater than 60% of the radiation emitted by the radiant body for at least one design wavelength.

In accordance with one optional feature which can be used with the above definition or with other features defined herein, the radiant body is a source of the radiation.

In accordance with one optional feature which can be used with the above definition or with other features defined herein, the radiant body is an emitting tube of the radiation.

In accordance with one optional feature which can be used with the above definition or with other features defined herein, the radiation source is in contact with at least one directing component.

In accordance with one optional feature which can be used with the above definition or with other features defined herein, at least one optical element of the optical directing components is integral with the radiation source.

In accordance with one optional feature which can be used with the above definition or with other features defined herein, all the radiation from the radiation source is collected and directed along a common path.

In accordance with one optional feature which can be used with the above definition or with other features defined herein, the radiant body is an object to be observed and the radiation is illuminating radiation from a separate source that is reflected from the radiant body. Preferably the object to be observed is moving along a path and the optical directing components surround the path. In one arrangement the illuminating radiation is transmitted along the path. Alternatively the illuminating radiation of the object is applied at angularly spaced positions around the path. In this arrangement, preferably the illuminating radiation at angularly spaced positions around the path is applied in a slightly divergent path terminated by beam stop which absorbs the radiation. In this arrangement, preferably the angularly spaced positions of the illuminating radiation around the path are arranged alternately with the optical directing components. In this arrangement, preferably the collected radiation is directed to a device for analyzing the collected radiation, for example a spectrometer.

In accordance with one optional feature which can be used with the above definition or with other features defined herein, at least one directing component is comprised of a plurality of optical elements. Preferably the optical elements are refractive or reflective.

In accordance with one optional feature which can be used with the above definition or with other features defined herein, a plurality of the optical directing components includes at least one common optical element.

In accordance with one optional feature which can be used with the above definition or with other features defined herein, the optical directing components are arranged to subtend substantially all of the solid angles into which radiation is emitted wherein each optical directing means subtends less than or equal to half of the emission solid angles.

In one arrangement the radiation source is an ionized gas.

In accordance with one optional feature which can be used with the above definition or with other features defined herein, the optical directing components are arranged symmetrically about an axis of the radiation source.

In accordance with one optional feature which can be used with the above definition or with other features defined herein, the path of directed radiation does not intersect the radiation source.

According to one aspect of the invention which can be used independently or in combination with any of the other features described herein there is provided a method for observing a three-dimensional radiant body comprising:
causing the radiant body to move along a path;
applying illuminating radiation to the radiant body while moving in the path;
collecting radiation reflected by the radiant body with a plurality of optical directing components;
arranging the optical directing components at angularly spaced positions around the path;
wherein each optical directing component subtends less than or equal to one half of the solid angle radiation is reflected by said three-dimensional radiant body.

In accordance with one optional feature which can be used with the above definition or with other features defined herein, the collected radiation is directed to at least one aperture where an area of the aperture is less than the surface area of the three-dimensional radiant body.

In accordance with one optional feature which can be used with the above definition or with other features defined herein, the aperture consists of a plurality of apertures and wherein the area of the apertures combined is less than the surface area of the radiation source.

In accordance with one optional feature which can be used with the above definition or with other features defined herein, each optical directing component subtends less or equal to one third of the solid angle radiation is reflected into by said three-dimensional radiant body

In accordance with one optional feature which can be used with the above definition or with other features defined herein, each optical directing component subtends less or equal to ⅙ of the solid angle radiation emitted by said radiant body.

In accordance with one optional feature which can be used with the above definition or with other features defined herein, there are six equiangular optical directing components around the path.

In accordance with one optional feature which can be used with the above definition or with other features defined herein, the optical directing components comprise wave guides.

In accordance with one optional feature which can be used with the above definition or with other features defined herein, the optical directing components comprise lenses.

In accordance with one optional feature which can be used with the above definition or with other features defined herein, the collected radiation is greater than 60% or the radiation reflected by the radiant body for at least one design wavelength. However this value can be higher.

In accordance with one optional feature which can be used with the above definition or with other features defined herein, the optical directing components are arranged to subtend substantially all of the solid angles into which radiation is reflected wherein each optical directing means subtends less than or equal to half of the reflected solid angles.

According to one aspect of the invention which can be used independently or in combination with any of the other features described herein there is provided a method for collecting radiation emitted from a three-dimensional radiation source comprising:
collecting radiation emitted by the radiation source with a plurality of optical directing components arranged at angularly spaced positions around the source;
transmitting the collected radiation to one or more end use locations;
wherein each optical directing component subtends less or equal to one half of the solid angle radiation is emitted into by said three-dimensional radiation source;
and wherein the collected radiation is greater than 60% of the radiation emitted by the radiation source for at least one design wavelength.

In accordance with one optional feature which can be used with the above definition or with other features defined herein, the radiation source has an axis of symmetry and the optical directing components are arranged symmetrically about the axis.

In accordance with one optional feature which can be used with the above definition or with other features defined herein, the collected radiation is directed to at least one aperture where an area of the aperture is less than a surface area of the three-dimensional radiation source.

In accordance with one optional feature which can be used with the above definition or with other features defined herein, at least one optical element of the optical directing components is integral with the radiation source.

In accordance with one optional feature which can be used with the above definition or with other features defined herein, all the radiation from the radiation source is collected and directed along a common path.

According to one aspect of the invention which can be used independently or in combination with any of the other features described herein there is provided a method for applying electromagnetic radiation to reactive materials in a reaction chamber comprising:
introducing the electromagnetic radiation into the chamber;
and increasing the probability of interaction of the electromagnetic radiation with the reactant materials by using multiple reflections to increase the optical path length of the electromagnetic radiation within the reaction chamber;
wherein the reaction chamber includes a plurality of pairs of opposed reflective surfaces of the chamber;
wherein at least 50% and more preferably at least 80% or 90% of the reflections from the reflective surfaces are specular reflections;
wherein at least one of the reflective surfaces of each pair is a concave mirror;
the reflective surfaces of each pair being arranged to cause reflections of the electromagnetic radiation back and forth between the reflective surfaces within a volume defined by the reflective surfaces;
the reflective surfaces of each pair being spaced one from the other so as to define a first side of the volume on one side of the reflective surfaces and so as to define a second side of the volume on an opposed side of the reflective surfaces;
wherein the pairs are arranged side by side so that radiation escaping through a side of one volume enters a side of a next adjacent volume.

In accordance with one optional feature which can be used with the above definition or with other features defined herein, the plurality of pairs define a stack of the volumes side by side where the radiation can pass between each volume and a next adjacent volume.

In accordance with one optional feature which can be used with the above definition or with other features defined herein, end ones of the volumes have a reflective side wall on an outer one of the sides thereof.

In accordance with one optional feature which can be used with the above definition or with other features defined herein, the reflective surfaces form side walls of a duct.

In one arrangement the flow is at right angles to the sides.

In one arrangement the radiation is directed into a duct through which a fluid passes.

In one arrangement the radiation is directed generally longitudinally of the duct.

In one arrangement the radiation is directed at an angle to a longitudinal direction of the duct with the radiation passing through a window in side walls of the duct.

In accordance with one optional feature which can be used with the above definition or with other features defined herein, a majority of radiation paths include at least ten and preferably more than one hundred reflections from surfaces bounding the reaction chamber. Put another way, the surfaces of the reaction chamber are arranged such that a majority of radiation paths are constrained to a volume within the reaction chamber and are incident on the reflective reaction chamber surfaces at least ten and preferably more than one hundred times before the radiation path exits the reaction chamber.

In accordance with one optional feature which can be used with the above definition or with other features defined herein, the reflective surfaces define at least one center optical axis extending therebetween along which the reflections pass and wherein a source of the radiation is located at a position offset from the center axis between the reflective surfaces so that a locus of the reflections moves toward the center axis.

In accordance with one optional feature which can be used with the above definition or with other features defined herein, a source of the radiation is located at one side of said at least one reflective surface of a reflective pair.

In accordance with one optional feature which can be used with the above definition or with other features defined herein, the reflective surface is a concave mirror and a source of the radiation source is located at a position on said at least one concave mirror and wherein the source of the radiation has a dimension which is less than 0.03 times the focal length of the mirror.

In accordance with one optional feature which can be used with the above definition or with other features defined herein, a source of the radiation source is located at a focal point of the concave mirror.

In accordance with one optional feature which can be used with the above definition or with other features defined herein, the offset between each beam and a next beam after a reflection is less than a width of the beam so that the beams form a complete curtain.

In accordance with one optional feature which can be used with the above definition or with other features defined herein, there is provided an inlet port for admitting reactive materials and an outlet port for discharging product materials and wherein there is provided absorbing surfaces formed and shaped to stop transmission of electromagnetic radiation from the interior of the chamber to an exterior location. In this arrangement, preferably the inlet and outlet ports are not on an axis of symmetry of the reaction chamber.

In accordance with one optional feature which can be used with the above definition or with other features defined herein, at least part of a chamber wall reflects electromagnetic radiation diffusely. This feature increases the homogeneity of the radiation field within the reaction chamber. Empirically, dust, manufacturing defects, and small scratches are sufficient to provide sufficient diffuse scattering (a few percent) for the homogenization purpose.

In accordance with one optional feature which can be used with the above definition or with other features defined herein, the reactive material is entrained in a fluid flow wherein the fluid is a liquid or a gas.

In accordance with one optional feature which can be used with the above definition or with other features defined herein, the electromagnetic radiation is UVC radiation and the reactive material is a microorganism selected from the list of bacteria, virus, protozoan, helminth, yeast, mold or fungus and said UVC radiation inactivates said microorganism. The UVC radiation is preferably comprises of radiation with wavelengths between 220 nm and 280 nm.

In accordance with one optional feature which can be used with the above definition or with other features defined herein, the electromagnetic radiation is at least partially collimated to travel primarily back and forth between the reflective surfaces.

According to one aspect of the invention which can be used independently or in combination with any of the other features described herein there is provided a method for applying electromagnetic radiation to reactant materials in a reaction chamber comprising:
introducing the electromagnetic radiation of a predetermined wavelength into the chamber;
and increasing the probability of interaction of the electromagnetic radiation with the reactant materials by using multiple reflections from reflective surfaces of the reaction chamber to increase the optical path length of the electromagnetic radiation through the reaction chamber for which the amplitude of the electromagnetic radiation is above a threshold value;
wherein at least one reflective surface of the reaction chamber comprises a metallic reflective wall at least part of which is coated with a layer of a material which has a high refractive index and has a low absorption of the radiation at the predetermined wavelength;
the layer of the material having a thickness selected to increase the reflectivity of the radiation at the reflective surface to a value greater than that of the metallic layer alone. In general, the amplitude of electromagnetic radiation scales as R^{N}, where R is the effective reflectivity and N is the number of reflections. The radiation field within the reaction chamber is calculated by dividing the reaction chamber into an array of small volume elements and summing the path amplitudes within each volume element. The threshold amplitude is chosen such that amplitudes below the threshold don't alter the calculated radiation field within a volume element by more than a tolerance amount. Empirically, for initial amplitude of 1.0000, threshold amplitude of 0.0001 was found to work well. Other threshold values may be used. In this arrangement, preferably the material is ZrO₂. In this arrangement, preferably the thickness of high refractive index material is varied at different locations on the surface. In this arrangement, preferably the thickness of high refractive index material is varied at different locations on the surface in dependence on an angle of incidence of the radiation on the surface so that the thickness of high refractive index material is increased at locations of greater angle of incidence. In this arrangement, preferably the high refractive index material is omitted so that the metallic wall is bare at locations of angle of incidence greater than a predetermined value. Preferably the wall is aluminum although other reflective materials can be used.

In this arrangement, preferably the material provides an increased hardness relative to the metallic wall.

According to one aspect of the invention which can be used independently or in combination with any of the other features described herein there is provided a method for collecting radiation emitted from a radially symmetric radiation source comprising:
providing first and second parabolic reflectors each having a reflective surface defining a rear neck and a forwardly projecting mouth;
locating the first and second parabolic reflectors back-to-back so that they intersect at the necks and the respective reflective surfaces extend from respective necks to the respective mouth;
locating the radiation source at the focal point of each of parabolic reflectors; and collecting the radiation emitted from the mouth of each parabolic reflector;
wherein the radiation emitted by the source in a direction away from the mouth of each reflector enters the other reflector so as to avoid radiation being reflected back to the source to be absorbed. In this arrangement, preferably the radiation from each reflector is collected separately. Alternatively the radiation from both reflectors is combined to be transmitted to a common end use location.

In this arrangement, preferably the source and the parabolic reflectors are symmetrical about a longitudinal axis.

In this arrangement, preferably there is provided an optical guide member located in each parabolic reflector at a position on an axis thereof spaced from the source so that radiation emitted in a direction beyond the mouth and thus missing the reflective surface is redirected. In this arrangement, preferably the optical guide member is a lens which collimates the radiation along the axis of the parabolic reflector. Alternatively the optical guide member is a mirror which redirects the radiation onto one or other of the parabolic reflectors.

In accordance with one optional feature which can be used with the above definition or with other features defined herein, the source is an emitting cylindrical tube.

Aspects of the present invention as defined above may provide solutions to the three limitations described above for an extended source. For illustrative purposes the method of the invention is described for a cylindrical source which may for example be a gas discharge lamp or a linear filament emitting radiation. The methods described herein may be applied without loss of generality to more complex geometries by either enclosing the more complex geometry in a simple virtual surface such as a cylinder or a sphere, or by applying the methods described herein to each volume element of the complex shape wherein each volume element is approximated as a simple primitive such as a cylinder or a sphere. The light source may for example be a grain kernel enclosed in a cylinder.

In accordance with an important feature of the invention, there is provided an aperture and a source of electromagnetic radiation wherein the surface area of the source of electromagnetic radiation is greater than or equal to the surface area of the aperture and at least some of the radiation supplied by the radiation source is directed away from the aperture. Specifically, the dot product between a vector directed from at least one point of origin of radiation on or within the radiation source to the center of the aperture with the vector direction of at least some radiation from that point is less than zero. For example, an atom in a gas discharge tube may emit radiation in all directions with equal probability. In this case half of the directions have a component opposite to the direction from the atom to the aperture and consequently have dot product less than zero. For example, the surface normal to a point on the surface of a grain kernel may be directed opposite to the direction from the surface point to the aperture. In this case the dot product between the aperture direction and radiation direction for all scattered or reflected radiation from said surface point is less than zero.

In one aspect of the invention as defined above, the solid angle subtended by the emitting area of the source is divided into a plurality of regions and an optical directing means is applied separately to each region. The optical directing means may be diffractive, reflective, refractive, or any combination thereof. The optical directing means may include a diffractive surface such as a Fresnel lens, a mirror, a lens, a wave guide, or any combination thereof. Each optical directing means receives radiation flux at an input surface with a first area and directs said radiation flux to an output surface with a second area.

In accordance with an important feature of the invention as defined above, there is provided a plurality of collection optical assemblies wherein each collection assembly receives radiation from the radiation source over a different range of solid angles through an input surface and wherein at least one collection optical assembly receives at least some radiation from a direction away from the aperture. That is, at least one collection assembly receives radiation for which the dot product of the radiation direction and aperture direction is less than one.

The term optical assembly herein refers to a set of one or more logical optical elements that operate together to transfer radiant energy from first spatial distribution of radiant energy at an input surface (proximate to the radiation source) to a second spatial distribution of radiant energy at an output surface (proximate to the aperture). The input and output surfaces are curvilinear and may or may not correspond with a material interface. Each logical optical element corresponds with a physical optical element. Each physical optical element may be included in a plurality of sets of logical optical elements. That is a single physical optical element may perform the same function for a plurality of sets of logical optical elements. The optical elements may be reflective, refractive, diffractive, or any combination thereof.

A simple example is a cylindrical emitter with a circular cross section with two optical collection assemblies 180 degrees apart. A first collection assembly receives flux emitted into angles between 0 and 180 degrees and produces a first (imperfectly) collimated beam directed at 90 degrees. A second collection assembly receives flux emitted into angles between 180 and 360 degrees and initially produces a second (imperfectly) collimated beam directed at 270 degrees. The first beam and second beam can be made collinear by including in either of the optical collection assemblies a prism, corner cube or like optical element to rotate the direction of the corresponding beam by 180 degrees. In this example all of the flux is at least imperfectly collimated and none of the flux is reflected toward the source and absorbed. However, aberrations increase with the angular range accepted by optical elements within the directing means. Although the aberrations can be at least partially compensated by using multiple lenses in each optical collection assembly, the cost and optical losses both increase with this approach. Therefore it is preferable to divide the emitting angular range into smaller angular ranges, for example six (6) angular ranges of 60 degrees each.

In accordance with an important feature of the invention as defined above, a plurality of collection optical assemblies direct radiation from the radiation source to a common photoreaction chamber. In some embodiments each collection optical assembly directs radiation from the radiation source to separate ports on the common photoreaction chamber. In some embodiments a plurality of collection optical assemblies direct radiation from the radiation source to a common port on a common photoreaction chamber.

In accordance with an important optional feature of the invention as defined above, each logical collection optical assembly includes a logical collimation sub-assembly that operates to reduce the angular divergence of radiation at the output surface of the logical collection optical assembly.

In accordance with an important optional feature of the invention as defined above, a plurality of logical collection optical assemblies include a logical collimation sub-assembly that corresponds to the same physical collimation optical assembly. That is the physical collimation assembly combines radiation from a plurality of logical collection assemblies and outputs a radiation beam with less angular divergence than the inputs from the collection assemblies.

According to one aspect of the invention which can be used independently or in combination with any of the other features described herein there is provided a method for decontaminating a body after potential contamination by one or more pathogens comprising:
generating a beam of radiation arranged to deactivate the pathogen;
applying the beam to the body
and controlling the beam to apply different doses of the radiation in the beam to different locations on the body depending on properties of the body at the different locations.

In accordance with one optional feature which can be used with the above definition or with other features defined herein, the different doses of radiation applied by the beam to different locations are calculated based on the concentration of pathogens present at each location and the probability that pathogens will be transferred from each location to a host species wherein the dose at each location is selected to minimize the probability of transmission to a host species from all locations. The host species may for example be humans.

In accordance with one optional feature which can be used with the above definition or with other features defined herein, the different doses of radiation applied by the beam to different locations are calculated based on the total dose available for all locations, the concentration of pathogens present at each location, and the probability that pathogens will be transferred from each location to a host species wherein the dose at each location is selected to minimize the probability of transmission to a host species from all locations subject to a total dose constraint.

In accordance with one optional feature which can be used with the above definition or with other features defined herein, the different doses of radiation applied by the beam to different locations are calculated based on the concentration of pathogens present at each location and the probability that pathogens will be transferred from each location to a host species wherein the dose at each location is selected to reduce the pathogen concentration at each location below a pre-determined threshold concentration.

In accordance with one optional feature which can be used with the above definition or with other features defined herein, the initial pathogen concentration at each location is estimated by a statistical model based on previously measured pathogen concentrations for that location and the dose is calculated to reduce said initial pathogen concentration below a pre-determined threshold value.

In accordance with one optional feature which can be used with the above definition or with other features defined herein, the pre-determined threshold pathogen concentration for each location is based at least in part on the probability of transmission from said location to a host species. The probability of transmission for each location may be based on empirical measurements for that location or statistical inference from known transmission cases.

In accordance with one optional feature which can be used with the above definition or with other features defined herein, the beam is controlled based on the reflectivity, scatter and absorbency as functions of the angle of incidence at the location.

In accordance with one optional feature which can be used with the above definition or with other features defined herein, the beam is controlled based on information about dose sensitivity at the location.

In accordance with one optional feature which can be used with the above definition or with other features defined herein, the beam is controlled based on the probability of contamination at that location and proximate locations.

In accordance with one optional feature which can be used with the above definition or with other features defined herein, there is provided a sensor to measure at least one of the temperature, pressure, humidity and molecular composition of fluid between a surface location and the source.

In accordance with one optional feature which can be used with the above definition or with other features defined herein, the beam is controlled based on the probability that contamination at that location has been transmitted to a second surface at the location by contact.

In accordance with one optional feature which can be used with the above definition or with other features defined herein, the beam is controlled based on the probability that contamination at that location can be transmitted to a second surface.

In accordance with one optional feature which can be used with the above definition or with other features defined herein, the beam is controlled based on an input of a pathogen reduction target from a user.

In accordance with one optional feature which can be used with the above definition or with other features defined herein, the beam is controlled based on a human operator moving and orienting the beam in response to instructions and feedback from a control system

In accordance with one optional feature which can be used with the above definition or with other features defined herein, the beam is controlled based on the position of a robotic platform that guides the decontamination system along a controlled path relative to the body to be decontaminated.

In accordance with one optional feature which can be used with the above definition or with other features defined herein, the beam is controlled based on subsystems that measure the position and orientation of the beam relative to the body that is to be decontaminated.

In accordance with one optional feature which can be used with the above definition or with other features defined herein, the beam is controlled based on measured the source intensity. That is the source intensity is measured and the time the beam is directed at each location on the body to be decontaminated is calculated based on the source intensity to provide a threshold dose of radiation.

In accordance with one optional feature which can be used with the above definition or with other features defined herein, the beam is controlled based on a measure of radiation reflected or scattered from a surface location. That is the reflected or scattered radiation is a known fraction of the incident radiation and the time the beam is directed at each location on the body to be decontaminated is calculated to deliver a at least a threshold dose based on the measured radiation scattered or reflected and the known fraction of scattered or reflected radiation collected. In some embodiments, the measured radiation may have a different wavelength than the radiation used for decontamination. For example, the measured radiation may be blue with wavelength 470 nm and the decontamination radiation may be UVC with wavelength 270 nm.

In accordance with one optional feature which can be used with the above definition or with other features defined herein, the method includes collecting a multi-spectral image of at least a portion of the body; determining at least in part the type of contamination present at different locations on said body by analyzing said multi-spectral image to determine the region of the multi-spectral image that corresponds to each location on the body; comparing the spectrum from a region of the multi-spectral image corresponding to each location on the body with reference spectra to determine the type of contamination present at each location; and directing the beam to each location on the body based at least in part on the type of contamination present at the location.

In accordance with one optional feature which can be used with the above definition or with other features defined herein, the method includes collecting samples from surface locations before or after irradiation and the samples collected are analyzed for viable pathogens.

According to one aspect of the invention which can be used independently or in combination with any of the other features described herein there is provided a method collecting samples from surface locations before or after irradiation by an agitator and a collector.

In accordance with one optional feature which can be used with the above definition or with other features defined herein, the agitator and collector are used to randomly sample locations on the surface of object and collected material is analyzed to provide detailed information about materials and contaminants present at that location.

In accordance with one optional feature which can be used with the above definition or with other features defined herein, the information from randomly selected locations is used to build statistical models to detect systemic problems with sanitization procedures.

In accordance with one optional feature which can be used with the above definition or with other features defined herein, the information from randomly sampled locations is used to build statistical models that predict the probability of contamination as a function of location.

In accordance with one optional feature which can be used with the above definition or with other features defined herein, the location dependent probability is used to optimize allocation of UVC dose

In accordance with one optional feature which can be used with the above definition or with other features defined herein, there is provided a position verification system as a component of the source. The position verification system operates to determine the position and orientation of the source relative to the body to be decontaminated.

In accordance with one optional feature which can be used with the above definition or with other features defined herein, the position verification system comprises a camera operating together with software to track as a function of time the locations and directions of the source.

In accordance with one optional feature which can be used with the above definition or with other features defined herein, a plurality of sample locations include a pattern of markings that are used by the position verification system to compute the location and orientation of the source.

In accordance with one optional feature which can be used with the above definition or with other features defined herein, the position verification system includes a device which measures the distance from the source to a surface location.

In accordance with one optional feature which can be used with the above definition or with other features defined herein, information is displayed on an overlay image to an operator wherein the overlay image contains an image of the surface to be decontaminated together with suitable representations of the dose received at each location relative to the dose required at each location.

According to one aspect of the invention which can be used independently or in combination with any of the other features described herein there is provided a method applying electromagnetic radiation to liquid flow in a chamber comprising:
introducing the electromagnetic radiation into the chamber;
and increasing the probability of interaction of the electromagnetic radiation with the reactant materials by using multiple reflections to increase the optical path length of the electromagnetic radiation within the reaction chamber;
wherein the reaction chamber includes at least one pair of opposed reflective surfaces of the chamber;
wherein at least 50% and more preferably at least 80% or 90% of the reflections from the reflective surfaces are specular reflections;
wherein at least one of the reflective surfaces of each pair is a concave mirror;
the reflective surfaces of each pair being arranged to cause reflections of the electromagnetic radiation back and forth between the reflective surfaces within a volume defined by the reflective surfaces;
wherein the chamber is an attachment adapter for attachment to a source of the liquid.

In accordance with one optional feature which can be used with the above definition or with other features defined herein, the adapter comprises a dispensing nozzle.

In accordance with one optional feature which can be used with the above definition or with other features defined herein, the adapter includes one or more couplings which are arranged for attachment to different sources. For example the adapter may include a threaded section, a press fit or a clip.

In accordance with one optional feature which can be used with the above definition or with other features defined herein, the adapter includes a filter.

According to one aspect of the invention which can be used independently or in combination with any of the other features described herein there is provided a method applying electromagnetic radiation to liquid flow in a chamber comprising:
introducing the electromagnetic radiation into the chamber;
and increasing the probability of interaction of the electromagnetic radiation with the reactant materials by using multiple reflections to increase the optical path length of the electromagnetic radiation through the reaction chamber;
wherein the chamber is arranged to association with a filter for attachment to a source of the liquid.

### BRIEF DECRIPTION OF THE DRAWINGS

FIG. 1 is a schematic cross-sectional view of a prior art parabolic reflector.

FIG. 2 is a schematic cross-sectional view of radiation directed from a cylindrical source into a single direction with lenses.

FIG. 3 is a schematic cross-sectional view of radiation from a cylindrical source into a single direction with an array of lenses and mirrors.

FIG. 4A is a schematic side view of an arrangement for collecting radiation scattered by an object in a tube.

FIG. 4B is a schematic side view of an arrangement for collecting radiation radially reflected and scattered by an object in a tube into a single direction with waveguides.

FIG. 5 is a schematic view of an arrangement for directing radiation onto an object in a tube and collecting reflected and scattered radiation for measurement.

FIG. 6A is a schematic cross-sectional view of an arrangement for directing radiation from a cylindrical source into a single direction with a compound parabolic reflector and lenses.

FIG. 6B is a schematic cross-sectional view of an arrangement for directing radiation from a cylindrical source into two directions with the compound parabolic reflector of FIG. 6A and lenses.

FIG. 7 is a schematic cross-sectional view of an arrangement for directing radiation from a cylindrical radiation source into a photochemistry reaction chamber through two ports using the compound parabolic reflector of FIG. 6A.

FIG. 8 is a schematic cross-sectional view of an arrangement for directing radiation into a chamber for fluid flow which can use the discharge arrangement of FIG. 7 or can use a cylindrical source at the focal point of one of the concave reflective mirrors.

FIG. 8A is an alternative schematic cross-sectional view similar to that of FIG. 8 of an alternative arrangement for directing radiation into a chamber for fluid flow.

FIG. 9 is a cross-sectional view through a portion of the wall of the chamber of FIG. 8 or FIG. 8A showing a further aspect according to the invention where the wall is coated with a reflective layer of ZrO₂.

FIG. 10 is a graph showing the effect on reflectivity of the arrangement of FIG. 9 where the wall is coated with a reflective layer of ZrO₂.

FIG. 11 shows an arrangement using the directed decontamination beam of FIG. 3 to effect decontamination of a body such as a vehicle seat between uses by different passengers.

FIGS. 12 and 13 are flow charts showing operation of the control system of the arrangement of FIG. 11.

FIG. 14 schematically shows an arrangement for sterilizing a flow of water.

### DETAILED DESCRIPTION

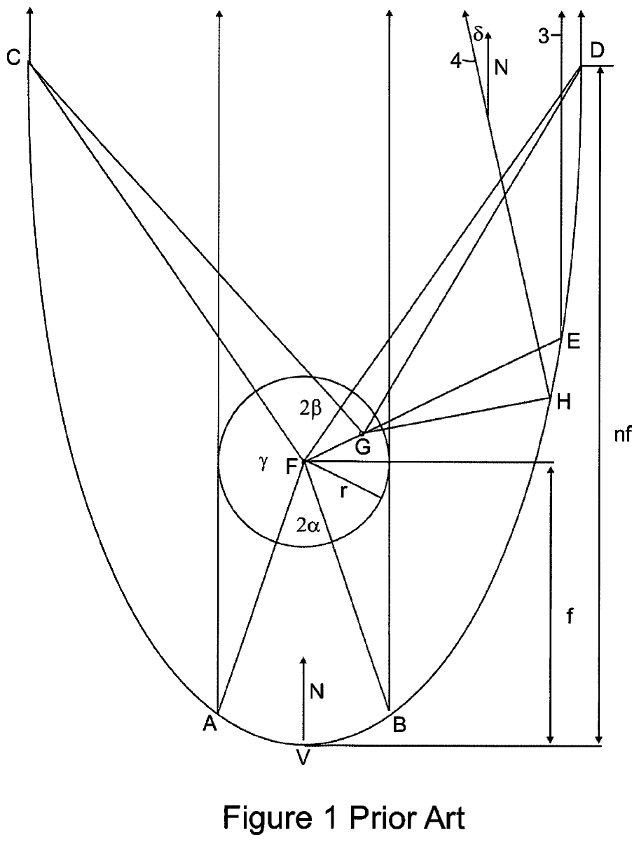

FIG. 1 schematically illustrates the characteristics of a prior art parabolic reflector. A radially symmetric radiation source 1 with radius r is positioned at the focal point F of parabolic reflector 2. The distance from the vertex V to the focal point F is f and the height of the parabolic reflector expressed in units of f from vertex V to edge D is nf, where n is a real number greater than one. Rays emitted radially from radiation source 1 are reflected by parabolic reflector 2 parallel to the parabola axis N. Rays emitted radially within the angle 2α defined by the points AFB are reflected by parabolic reflector 2 to radiation source 1 and suffer absorption. For a radially symmetric source, the fraction of energy lost to absorption is kα/π, where k is a geometrically averaged absorption constant and

$$α = {tan}^{- 1} ( r / f ) .$$

Rays emitted radially by radiation source 1 into the angle 2β defined by the points CFD are not incident on parabolic reflector 2 and hence are not collimated in the direction of parabola axis N. The angle β is given by

$$β = {tan}^{- 1} [ 2 {n}^{1 / 2} / ( n - 1 ) ] .$$

Rays emitted from radiation source 1 incident on parabolic reflector in the angle γ=π−α−β defined by the points AFC are reflected substantially parallel to parabola axis N.

For a general point G inside radiation source 1, rays emitted radially along the line FE are reflected parallel to parabola axis N as shown at 3 and rays emitted toward a general point on the parabolic reflector H are reflected in the general direction of N with angular divergence δ from the direction of N increasing in general as the ratio r/f increases. Hence for small r/f rays are well collimated and for r/f large collimation is poor. Rays generally parallel to parabola axis N may be focused to form an image on an aperture plane with perfectly collimated rays imaged to a point at the center of the aperture and rays with increasing angular divergence δ imaged increasingly far from the aperture center. The fraction not focused on the aperture is a function h (r/f). That is the required aperture size increases with angular divergence and r/f ratio. Further, most of the rays emitted into the angle 2β have large divergence from parabola axis N. Although rays in the angle 2β may be imaged onto an aperture, the focal length of the focusing element is different from the focal length required to image rays in the angle γ onto the aperture. The total optical loss of the prior art system is hence approximately

$$Loss = ( α + β + h ( r / f ) γ ) / π .$$

Arrangements of the present invention discussed below eliminate the α and β terms and reduce the magnitude of the γ term.

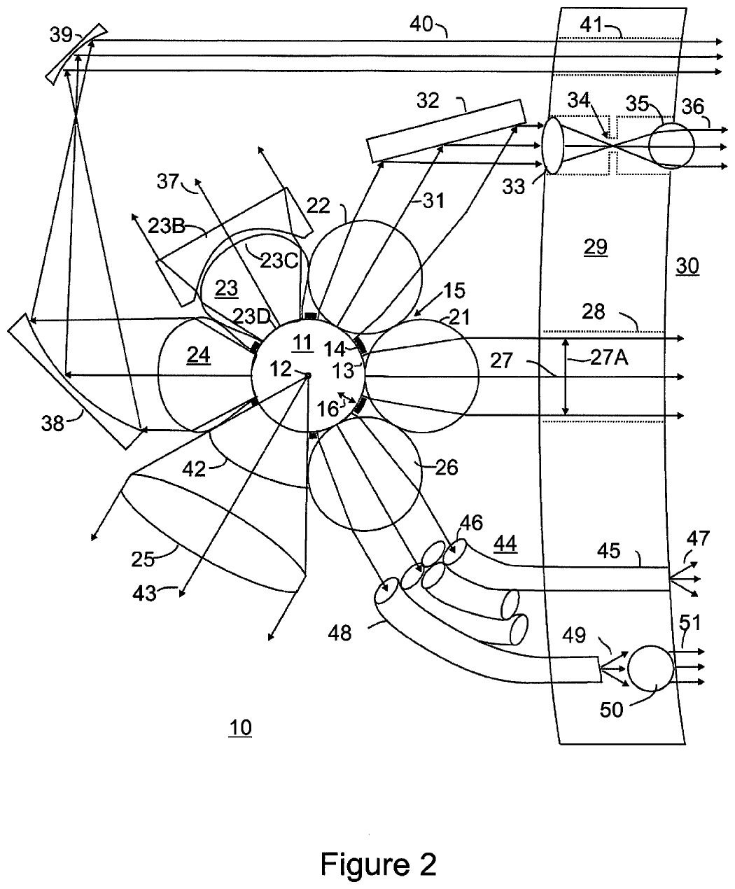

FIG. 2 schematically illustrates six optical embodiments of the invention generally indicated at 10. That is, in the single drawing, six different options are illustrated at the six locations around the axis of the source. In practice a practical embodiment will use the same option at each location, but this is not included as a separate figure for each option for convenience of illustration.

Cylindrical tube emitter 11 has axis 12 perpendicular to the plane of the illustration and is bounded by transparent container 13. Container 13 may include reflective sections 14 positioned at the intersection of optical regions 15 that reflect radiation back into tube emitter 11 as shown at 16. Tube emitter 11 is surrounded by six optical lenses 21, 22, 23, 24, 25 and 26. Preferably the lenses are anti-reflection coated to reduce Fresnel reflection losses. For illustrative convenience each lens subtends an equal angle, but there is no requirement for the subtended angles to be equal. In some embodiments each subtended angle may be different.

Lens 21 may be a cylindrical lens that receives radiation from source 11 and forms collimated beam 27 that passes directly through aperture 28 in chamber wall 29. A cylindrical lens is simple to fabricate and collimates radiation incident on near the optical axis well, but suffers from aberration at the edges. As shown radiated power incident near the optical axis is collimated and beam 27 passes directly through aperture 28 into chamber 30. The width of beam 27 indicated at 27A is less than the diameter of tube 25 emitter 11. In some embodiments, as indicated at 14, a reflective coating may be placed on or proximate to tube emitter 11 over a small region near the junction between two lens sectors 15. The reflective regions 14 have angular extent just sufficient to intercept radiation that would not be properly focused onto an aperture at the periphery of optical elements due to aberration. A fraction of the power reflected by reflective regions 14 is re-emitted in a random direction with high probability of being re-emitted in a direction that is properly focused to an aperture. In embodiments that use lenses designed to correct for aberration, the reflective regions may be omitted.

Lens 22 collects radiated power from cylinder tube emitter 11 and directs collimated beam 31 incident onto fold mirror 32. Fold mirror 32 redirects the collimated beam along a desired optical axis perpendicular to chamber wall 29 and onto focusing lens 33. Focusing lens 33 focuses the collimated beam through aperture 34 and radiation passing through aperture 34 is re-collimated by lens 35 and enters chamber 30 as collimated beam 36. The beam diameter of re-collimated beam 36 is a fraction of the beam diameter of beam 31, which in turn is less than the diameter of emitting tube 11. That is the emission from tube 101 in the direction of lens 22 is compressed to an area substantially smaller than the dimensions of emitter tube 11. Note that the beam divergence of beam 36 is increased in proportion to the ratio of beam diameter 31 to beam diameter 36. In embodiments where aperture 34 and chamber 30 are elements of the reaction chamber for sterilization described in the above cited MPS patent by the present inventors, a beam divergence below a threshold value is acceptable and even slightly advantageous insofar as the increased beam divergence reduces the probability of radiation being reflected within the chamber back through aperture 34. The threshold beam divergence is selected such that most of the radiant power of a beam passing through aperture 34 is directly incident upon a highly reflective concave end mirror (not shown) of reaction chamber 30. Radiative power incident on the concave end mirror is constrained by the chamber geometry to propagate mainly along the chamber optical axis.

Lens 23 and 23B schematically illustrate that a multi-lens system may be used to correct for aberration and collimate radiated power incident over an increased angular range. As shown at 23C and 23D, the surfaces of lens 23 are non-cylindrical. Lens surfaces 23C and 23D are shaped to work with additional lenses 23B to increase the numerical aperture and to reduce the angular divergence of collimated radiation. The collimated beam 37 may be directed through an aperture for example with a folding mirror (not shown) as illustrated for the optical path beginning with lens 22 described above.

Lens 24 and concave mirrors 38 and 39 schematically illustrate an alternative arrangement to the arrangement shown with lens 22 for projecting radiation through a small aperture. Lens 24 is shaped to accept and collimate radiated power over a wider angular range than a cylinder lens. Concave mirror 38 focuses radiation collected and collimated by lens 24 and the focused radiation is re-collimated by concave mirror 39 to form collimated beam 40 that passes through aperture 41 into chamber 30. In the arrangement shown, concave mirrors 38 and 39 operate to rotate the direction of the radiation beam by 180 degrees and to magnify the beam diameter by a factor of less than one in the ratio of their focal lengths. Preferably mirrors 38 and 39 are high reflectance dielectric mirrors with reflectivity optimized for the average angle of incidence (45 degrees as shown) as described in the above cited MPS application. Different angles of incidence may be used with dielectric mirrors optimized for the different angles of incidence.

Lens 25 is displaced from emitter tube 11 and subtends the entire 60 degree angle of the sector as shown at 42. By increasing the distance, between the emitter tube and lens, a longer focal length lens may be used with less aberration at the edges. Lens 25 produces collimated beam 43 with greater width than the diameter of emitter tube 11. Beam 43 may be reduced in diameter and then directed through an aperture as illustrated for the optical paths beginning with lenses 22 and 24. Specifically, plane mirror 32 is oriented to geometrically reduce the width of (imperfectly) collimated beam 31. Concave mirrors 38 and 39 function to magnify the (imperfectly) collimated beam from lens 24 by a magnification factor less than one.

Lens 26 collects radiated power from emitter tube 11 and directs collimated radiation onto fiber optic array 44. Individual optical fibers may transmit radiation to any location on chamber wall 29. In a first fiber optic embodiment, collimated radiation enters fiber 45 at 46 and is transmitted to chamber 30 where the radiation is emitted with angular divergence corresponding to the fiber numerical aperture as shown at 47. In this case the angular divergence of radiation delivered to chamber 30 can be controlled by selecting an appropriate numerical aperture fiber. In a second fiber optic embodiment, collimated radiation enters a fiber 48 and is emitted at 49 with angular divergence determined by the numerical aperture of fiber 48. Radiation emitted at 49 is re-collimated by ball lens 50 and collimated beam 51 enters chamber 30. A larger numerical aperture fiber may be selected in this case because the collimation at the chamber is determined by the ball lens.

The embodiment of FIG. 2 therefore provides a method for collecting radiation emitted from a three-dimensional body defined by source 11 where radiation emitted by the body is collected with a plurality of optical directing components 21 to 26 where each optical directing component subtends less or equal to one half of the solid angle radiation is emitted into by said three-dimensional body.

As shown, the body or source 11 has an axis of symmetry and the optical directing components 21 to 26 are arranged symmetrically about the axis.

As shown, the collected radiation is directed to at least one aperture 41 where an area of the aperture is less than a surface area of the three-dimensional body.

As shown there can be a plurality of apertures where the total area of the apertures combined is less than the surface area of the radiation source.

As shown, each optical directing component subtends less or equal to one third of the solid angle radiation is emitted into by said three-dimensional body

As shown, each optical directing component subtends less or equal to ⅙ of the solid angle radiation is emitted into by said three-dimensional body.

As shown, there are six equiangular optical directing components around the body.

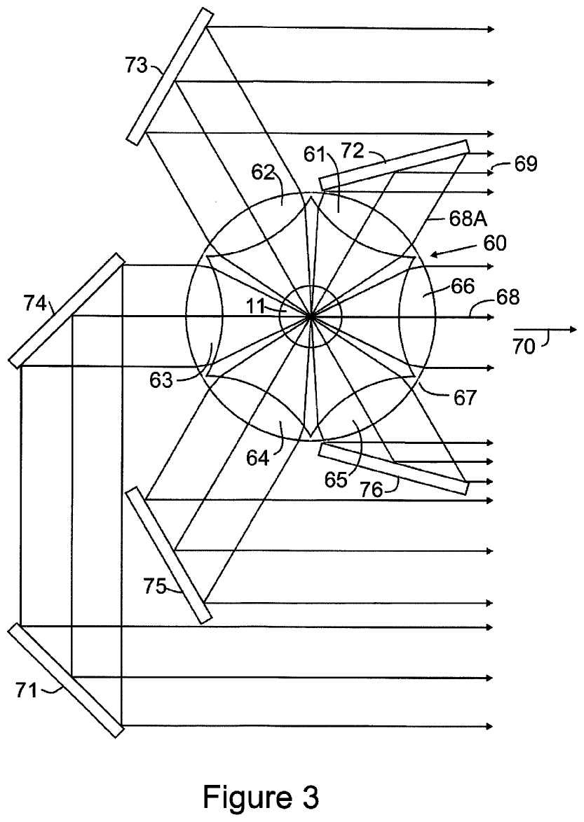

FIG. 3 shows a symmetric arrangement used to direct substantially all of the radiation emitted, scattered or reflected from source 11 in one direction as indicated at 70. An array 60 of lenses 61, 62, 63, 64, 65 and 66 are arranged radially about axis 12 of source 11 in equal angular increments. For illustrative purposes, an array of 6 lenses is shown. The number of lenses may be as few as 3 or as many as 36 or more. Preferably the number of lenses in the array is between 6 and 12. The lenses may be separate pieces mounted abutting in a support structure (not shown) or fabricated as a single piece. The lens array 60 is preferably mounted in a frame 67 that centers lens array 60 about axis 11 and allows translation along axis 12 for the purpose of accessing and maintaining source 11. Preferably each lens is positioned at a radial distance from axis 11 of between 2 and 3 times the radius of source 11. As the radial distance increases, the angular divergence of rays relative to each lens axis decreases leading to improved collimation of the output beam of each lens. As the radius of lens array 60 increases, the volume of lens material required increases leading to higher cost. Empirically the best comprise between angular divergence and cost is a lens array radius of about 2.4 times the source radius.

Each lens produces a collimated beam. Lens 66 produces collimated beam 68 with less angular divergence than rays incident on lens 66. In the embodiment shown the rays in beam 68 are nearly parallel. In an alternate embodiment (not shown), the rays in beam 68 may converge and a second optical element, for example a mirror, is positioned along the beam axis with curvature and position designed to produce a beam of lesser width. The beam width reduction is proportional to the ratio of focal lengths of the mirror and lens. Lens 61 receives radiation from source 11 and produces collimated beam 68A incident on mirror 72 which produces reflected beam 69 in the direction of axis 70. Mirror 76 and lens 65 are symmetric and equivalent to lens 61 and mirror 72. Likewise lenses 62 and 64 together with mirrors 73 and 75 form a symmetric pair that produces (as shown) increased beam width. Preferably this pair is in the second embodiment in which the lens and mirror curvatures interact to produce a beam with lesser beam width. As shown the beam from lens 63 is anti-parallel to axis 70 and the direction is brought into alignment with axis 70 using two folding mirrors 74 and 71. Hence radiation from source 11 is divided into six parts and each part is collimated and brought into alignment with axis 70. In practice, the arrangement shown brings more than 50% or 80% or 90% of the radiation from source 11 into alignment with axis 70 with divergence less than 5 degrees. In comparison a prior art parabolic reflector collimates approximately 55% of radiation within 5 degrees of the axis.

In some embodiments, the directional beam produced by the arrangement of FIG. 3 may be used to concentrate exposure to radiation produced by source 11 to a defined area. For example, the directional beam may be ultraviolet radiation used to decontaminate a surface with variable height relative to beam axis 70. In the arrangement shown the dose received is substantially independent of height (neglecting atmospheric absorption and residual beam divergence). The arrangement may be used for example to sanitize seats in a transport vehicle. For example, the arrangement in FIG. 3 may be used to increase the radiation flux from an infrared source projected onto a sample material for subsequent spectroscopic analysis. The sample material may for example be a grain kernel. Preferably the spectroscopic analysis is done with a spectrometer based on the above cited HEMS patent by the current inventors. In this case the increased photonic efficiency is compounded with the increased photonic efficiency of the HEMS spectrometer to give an improved signal-to-noise ratio.

In an alternate embodiment, the arrangement shown in FIG. 3 further includes a focusing element such as a lens or mirror (not shown) that focuses the six beams through an aperture. The aperture may for example be in the wall of a photochemical reaction chamber as discussed in the above cited MPS patent by the current inventors.

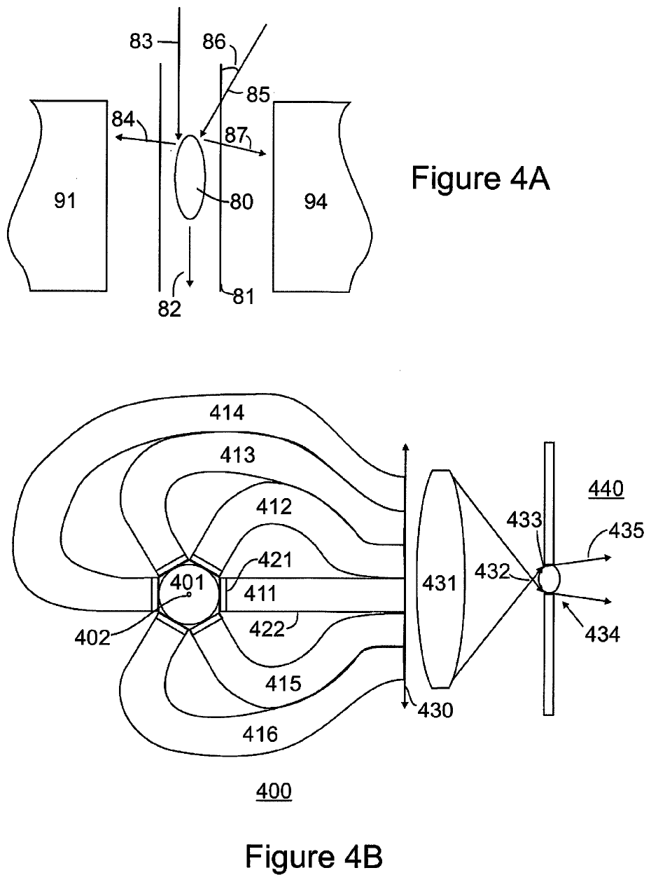

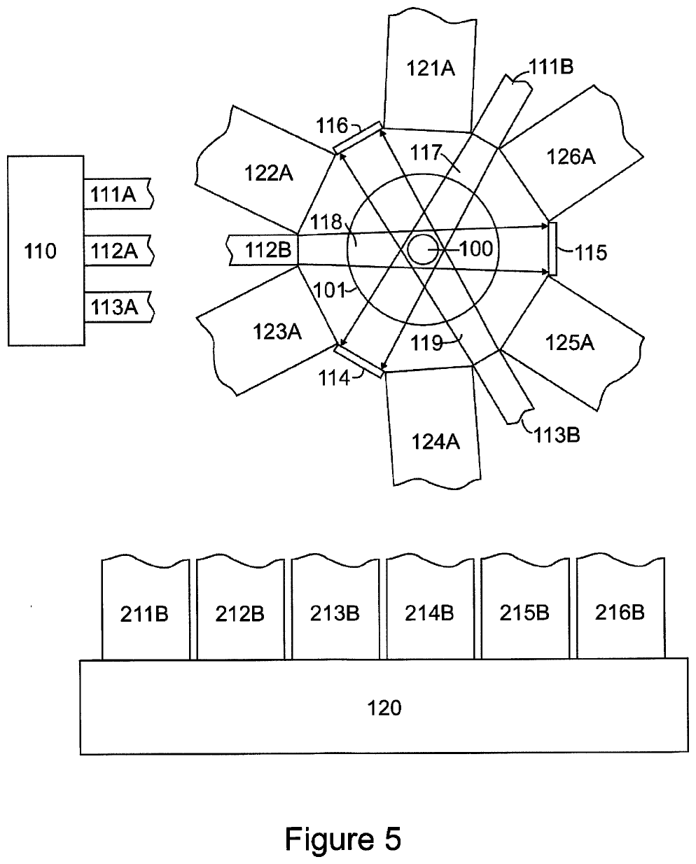

FIG. 4A shows the side view of an arrangement to illuminate an object in a tube and collect radiation scattered and reflected from the object. The tube may for example be the tube conveying a grain kernel. Kernel 80 is enclosed in transparent cylindrical tube 81 and travels in the direction of the tube axis as shown at 82. The tube may for example be quartz, fused silica or sapphire. The kernel may be illuminated by probe radiation along the tube axis as shown at 83 and reflected and scattered radiation 84 is incident on waveguide 91. The probe radiation may for example be near infrared radiation at wavelengths between 0.8 microns and 3 microns which are transmitted through quartz, fused silica or sapphire. Alternately, probe radiation 85 may be incident on tube 81 at angle 86, pass through the tube wall and be incident on kernel 80. Radiation 87 scattered or reflected from kernel 80 is incident on waveguide 94. Radiation incident on waveguides 91 and 94 is transmitted to a common output port for measurement.

FIG. 4B shows an arrangement generally indicated at 400 with a radiation source 401 radially symmetric about axis 402. Radiation source 401 is abutted by six waveguides 411, 412, 413, 414, 415, and 416 which are spaced at equal angles and form a hexagonal ring completely surrounding source 401. The waveguides optionally include an anti-reflection coating 421 that is designed to reduce Fresnel reflection at the waveguide interface for a range of design wavelengths. For example, the radiation source 401 may be a mercury vapor gas discharge tube that emits radiation centered at a wavelength of approximately 256 nm. For example the waveguides may be fabricated with fused silica (SiO₂), which has a refractive index of approximately 1.498 at 256 nm. Radiation incident from the source at every angle of incidence is refracted into the waveguide and all of the refracted radiation is incident on waveguide side walls 422 at more than the critical angle provided that the curvature of the waveguide is kept below a threshold value. As shown in FIG. 4B, waveguides 412, 413, 414, 415 and 416 follow curved paths to a common output plane 430. The waveguide curvature as shown is more than the threshold value for illustrative purposes only. In practical embodiments the curvature is kept below the threshold value so that total internal reflection occurs and radiant energy is transmitted from the radiation source to plane 430 without loss. In embodiments in which the cross-sectional area of each waveguide is substantially constant (within manufacturing tolerances), the distribution of ray angles relative to the waveguide axis is the same at the input (source) end and output (plane 430) end. As the number of waveguides increases, the distribution of ray angles incident on each waveguide shifts to lower angles. Put another way, the angular divergence from the mean direction in each waveguide decreases as the number of waveguides increases (and the angular range of the source subtended by each waveguide decreases). Radiation emitted through the waveguide ends in plane 430 is focused by lens 431 onto aperture 434 through focus 432. A lens 433 placed in aperture 434 reduces the angular divergence of radiation emitted into photoreaction chamber 440 as shown at 435.

The arrangement shown in FIG. 4B, while shown for collating and directing light from a source, can also be used as in FIG. 4A with light emitted, reflected, or scattered by an object to be observed.

In FIGS. 2 and 3 therefore there is provided a method for collecting radiation emitted from a three-dimensional source 11 where radiation emitted by the source 11 is collected with a plurality of optical directing components 21 to 26 in FIGS. 2 and 61 to 66 in FIG. 3 arranged at angularly spaced positions around the source. The collected radiation is transmitted by one of a number of optional arrangements to one or more end use locations.

Each optical directing component subtends less or equal to one half of the solid angle radiation is emitted into by said three-dimensional body and this enables the collected radiation to be greater than 60% of the radiation emitted by the body for one or more design wavelengths. In some arrangements this enables 50% or 80% or 90% of the radiation from source 11 into alignment with axis 70 with divergence less than 5 degrees for one or more design wavelengths.

Typically the body has an axis of symmetry and the optical directing components 21 to 26 are arranged symmetrically about the axis.

In some cases the collected radiation is directed to a single aperture 41 where an area of the aperture is less than a surface area of the three-dimensional body.

In other cases the radiation is direct to a plurality of apertures and the area of the apertures combined is less than the surface area of the radiation source.

As shown in FIG. 2, each optical directing component subtends less or equal to one third of the solid angle radiation is emitted into by said three-dimensional body. In particular, each optical directing component subtends less than or equal to ⅙ of the solid angle radiation is emitted into by said three-dimensional body. Thus there are six equiangular optical directing components around the body.

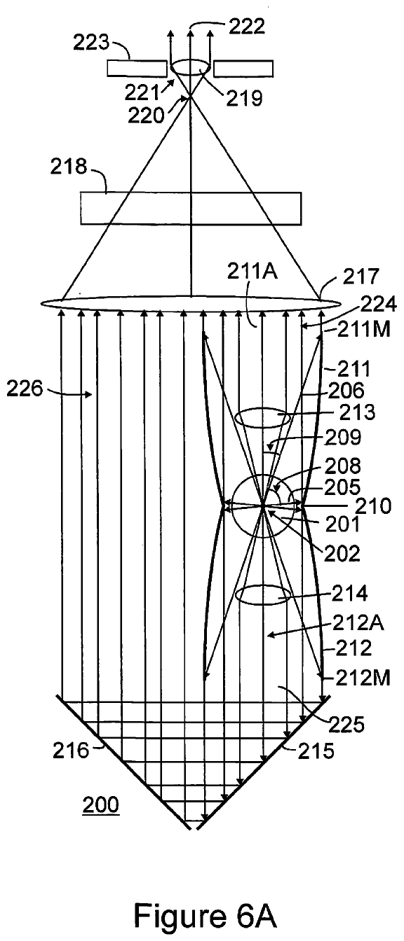

In FIGS. 4A and 5 therefore there is provided a method for observing a three-dimensional body where the body moves along a path and illuminating radiation is applied to the body while moving in the path.

The radiation reflected by the body is collected with a plurality of optical directing components where the optical directing components are arranged at angularly spaced positions around the path and each optical directing component subtends less than or equal to one half of the solid angle radiation is reflected by said three-dimensional body.

As above, each optical directing component subtends less or equal to one third of the solid angle radiation is reflected into by said three-dimensional body and typically less or equal to ⅙ of the solid angle radiation emitted by said body. Thus there are six equiangular optical directing components around the path.

FIG. 5 shows a further arrangement similar to that of FIG. 4A for directing radiation onto a singulated object 100 to be observed passing along a tube 101 and for collecting reflected and scattered radiation from the object for measurement. In this embodiment, the object is shown at 100 and is illuminated by three beams 117, 118 and 119 directed radially onto the object. Each beam 117, 118 and 119 is supplied by a respective light guide 111B, 112B and 113B, which carries light from a set of split sources 111A, 112A and 113A which may for example correspond to waveguides 411, 412, and 413 in FIG. 4. Alternately split sources 111A, 112A and 113A may each be generated by a suitable separate source. The light guides 111B, 112B and 113B may include a collimation lens at the terminal end (not shown). The guides are located at 120 degrees spacing around the tube 101. The guides can be sheets so that they have a length along the tube greater than the angular dimension. Each beam is this projected from the surface of the tube radially inwardly to pass through the axis of the tube where the object is preferentially located. Each beam terminates at a respective beam stop 114, 115, 116 which forms an absorbent material so that radiation passing the object to the other side of the tube is absorbed rather than reflected or scattered for collection. While perfect collimation of beams 117, 118 and 119 is desirable, it is impractical. The optical elements 111B, 112B and 113B are designed and fabricated to keep the beam divergence below a threshold angle. The threshold angle may for example be in the range of 3 to 5 degrees. As shown the beams are slightly divergent so the angle subtended by the stops is greater than the angle subtended by the respective emitting guide.

Light emitted from the object is collected by six collectors 121A to 126A which carry the light through light guides to the inputs 121B to 126B at the spectrometer 120. The spectrometer may for example be the above cited HEMS arrangement by the current inventors.

The collectors thus fill the spaces between the emitted and the respective beam stops so that the light is collected around the full 360 degrees apart from the angles subtended by the emitters and beam stops.

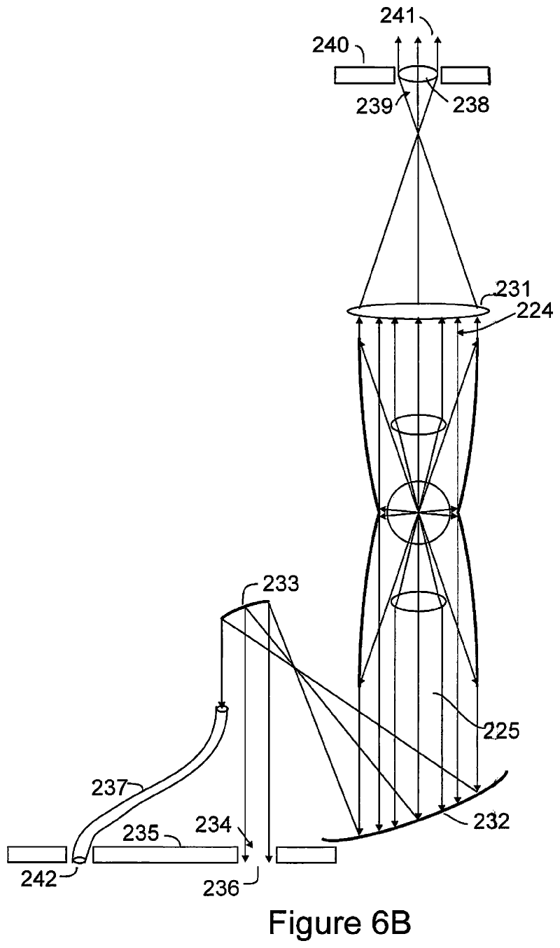

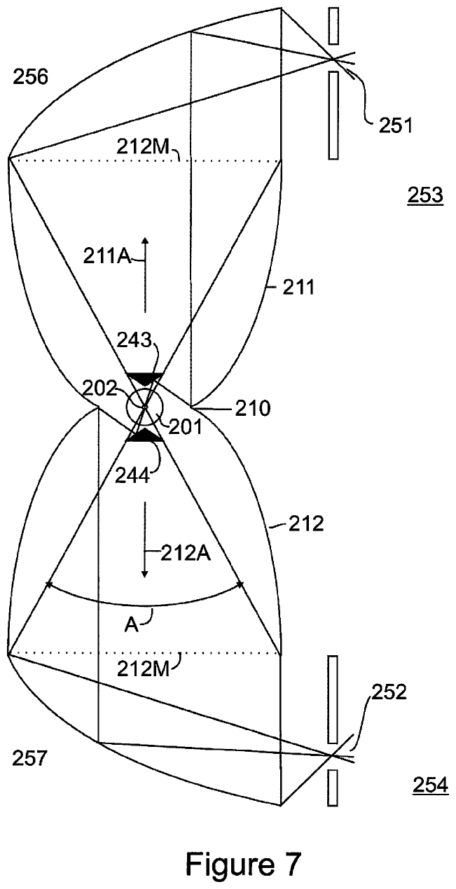

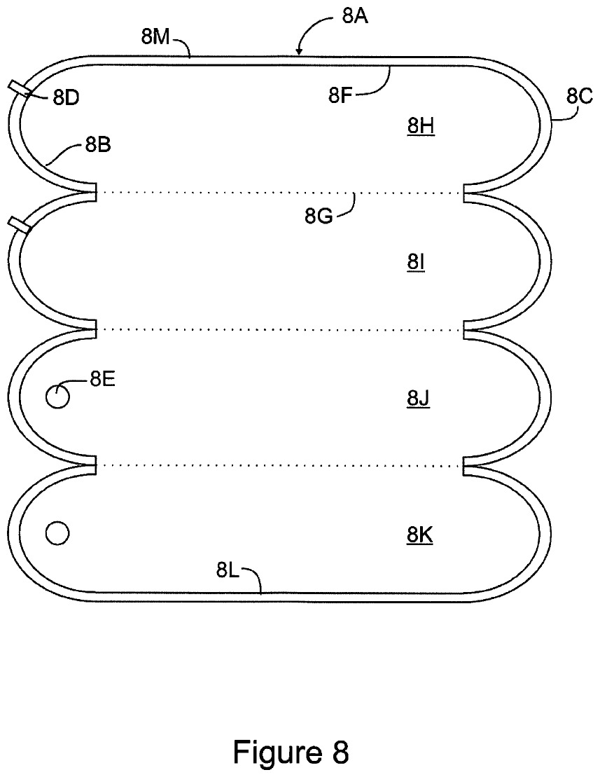

FIGS. 6A, 6B and 7 show another arrangement for use with a source which uses two parabolic reflectors in a back-to-back array. The source is typically elongate such as a tube.

FIG. 6A shows an arrangement for collimating and directing radiation from a radially symmetric radiation source through an aperture generally indicated at 200. Radiation source 201 is radially symmetric about axis 202 and is located at the focal point of parabolic reflector 211 and parabolic reflector 212. Radiation source 201 may for example be a cylindrical gas discharge bulb. Parabolic reflector 211 has axis 211A perpendicular to bulb axis 202. Similarly parabolic reflector has parabolic axis 212A perpendicular to bulb axis 202. Parabolic reflectors 211 and 212 intersect at a neck 210 and the respective reflective surfaces extend from the common neck 210 to two separate mouths 211M and 212M, respectively. Neither parabolic reflector has a surface between the parabola vertex and the neck 210. As shown at 205, rays emitted by source 201 that intersect parabolic reflector 211 at any point between the neck 210 and mouth 211M within angle 208 are collimated in the general direction of parabola axis 211A. Note that angle 208 extends from a line between the bulb center and the neck to a line between the bulb center and parabola mouth 211M. Collimation is only perfect for rays emitted at points on a straight line between the focal point of parabolic reflector 211 and the point of intersection with parabolic reflector 211. Rays incident on parabolic reflector 211 from source points not in line with the focus are only approximately collimated within a small angle range centered on parabola axis 211A. Rays emitted into the angular range between parabola axis 211A and the line from the focal point 202 to mouth 211M as indicated at 209 are incident on an additional lens 213. Lens 213 may for example be a cylindrical lens with optical axis parallel to parabola axis 211A and height axis parallel to and coextensive with the bulb axis 202. Lens 213 is displaced from bulb center 202 by the focal length of lens 213 and consequently collimates incident rays generally parallel to parabola axis 211A. Rays emitted within source 201 from points along a line from center axis 202 and lens 213 are collimated perfectly (for an ideal lens) and rays emitted from other points within source 201 and incident on lens 213 are directed within as small range of angles close to parabola axis 211A. Half angles 208 and 209 from parabola axis 211A to neck 210 sum to 90 degrees, hence parabolic reflector 211 and lens 213 receive and collimate incident radiation emitted into a 180 degree range of angles mainly in the direction of parabola axis 211A as beam 224. The angular divergence of beam 224 can be kept below a selected threshold by selection of the parabola and lens focal lengths relative to the radius of radially symmetric source 201.

The arrangement of FIG. 6A is symmetric. Rays emitted by source 201 incident on parabolic reflector 212 are collimated mainly in the direction of parabola axis 212A as beam 225. Rays emitted by source 201 and incident on lens 214 are collimated mainly in the direction of parabola axis 212A as beam 225. Hence the radiation collimated in the direction of parabola axis 212A is received from a 180 degree range of angles from source 201. Hence substantially all of the radiation emitted by source 201 is collimated: half in the direction of parabola axis 211A as beam 224 and half in the direction of parabola axis 212A as beam 225. Radiation beam 225 collimated in the direction of parabola axis 212A is reflected by folding mirrors 215 and 216 to form beam 226 parallel to parabola axis 211A and laterally displaced from parabola axis 211A. Laterally displaced beam 226 and 224 are combined by lens 217 and focused to a scaled source for lens 219 at focus 220. The scaled source at 219 is an image of source 201 (and its reverse side) scaled by a magnification factor M less than one. Optionally, lens 218 is a cylindrical lens that operates to reduce angular divergence in the direction of source axis 202 perpendicular to the view shown. As shown, lens 219 collimates incident radiation through aperture 221 in wall 223 to form beam 222. The angular divergence of beam 222 is increased relative to the angular divergence of the beam incident on lens 217 by 1/M. Hence the focal lengths of lenses 217 and 219 are selected to keep the angular divergence of beam 222 less than a threshold value.

FIG. 6B illustrates arrangements for directing portions of radiation from a radially symmetric source through a plurality of apertures 236, 239 and 242. The parabolic reflectors 211 and 212 as well as lenses 213 and 214 are identical to the arrangement shown in FIG. 2A and produce beams 224 and 225. In FIG. 6B beam 224 is focused by lens 231 to form an image for lens 238 in aperture 239 in wall 240. Lens 238 collimates radiation from the image to form beam 241. The focal lengths of lenses 231 and 238 are selected to keep the angular divergence of beam 241 less than a threshold value. Beam 225 is incident on concave reflector 232 which focuses incident radiation onto concave reflector 233 which re-collimates the radiation with magnification given by the ratio of focal lengths of concave reflectors 232 and 233. A portion of the resultant collimated beam is directed through aperture 234 in wall 235 as beam 236. A portion of the resultant collimated beam is incident upon and transmitted through fiber optic 237 to aperture 242 in wall 235.

FIG. 7 shows the same arrangement of back-to-back parabolas 211 and 212 connected at a neck 210 and containing source 202. In this embodiment the radiation from the two parabolas is directed through to apertures 251 and 252 into reaction chambers 253 and 254 by redirecting the radiation using curved mirrors 256 and 257 respectively which are shaped as an off-axis parabolic sheet so as to collimate the rays which are focused at the respective aperture.

In FIG. 7 the lens 213, 214 is replaced by a mirror 243, 244 shaped as an isosceles triangle with an apex facing toward the source. Reflective triangle 244 intercepts radiation emitted into the angle A and reflect the intercepted radiation toward reflective parabola surface 212. Likewise reflective triangle 243 reflects radiation toward reflective parabola surface 211. The angle A is the angle defined by the respective parabola mouths and axis 202. The reflective triangles are placed as close as possible to radiation source 201 so that radiation reflected toward the parabolic surfaces comes from points near the source. This minimizes the angular divergence of radiation collimated by the parabolic surfaces. Empirically triangle apex angles between 100 and 120 degrees were found to optimize the fraction of radiation collimated with angular divergence less than 13 degrees.

The arrangements in FIGS. 6A and 6B thus provide a method for collecting radiation emitted from a radially symmetric radiation source. The method includes providing first and second parabolic reflectors 211 and 212 each having a reflective surface defining a rear neck 210 and a forwardly projecting mouth 211M and 212M where the first and second parabolic reflectors are arranged back-to-back so that they intersect at the necks and the respective reflective surfaces extend from respective necks to the respective mouth.

The radiation source 201 is located at the focal point of each of parabolic reflectors and the radiation emitted from the mouth of each parabolic reflector is collected and where the radiation emitted by the source in a direction away from the mouth of each reflector 211, 212 enters the other reflector 212, 211 so as to avoid radiation being reflected back to the source to be absorbed.

In FIG. 6B the radiation from each reflector is collected separately.

In FIG. 6A the radiation from both reflectors is collated to be transmitted to a common end use location.

The source and the parabolic reflectors are symmetrical about a longitudinal axis.

In each parabola is provided an optical guide member 213, 205 located in each parabolic reflector at a position on an axis thereof spaced from the source so that radiation emitted in a direction beyond the mouth and thus missing the reflective surface is redirected.

In FIGS. 6A and 6B the optical guide member is a lens which collimates the radiation along the axis of the parabolic reflector.

In FIG. 7 the optical guide member is a mirror which redirects the radiation onto one or other of the parabolic reflectors.

In FIG. 6A the collected radiation is directed to one aperture where an area of the aperture is less than a surface area of the source.

In FIGS. 6B and 7 there is a plurality of apertures and the area of the apertures combined is less than the surface area of the radiation source.

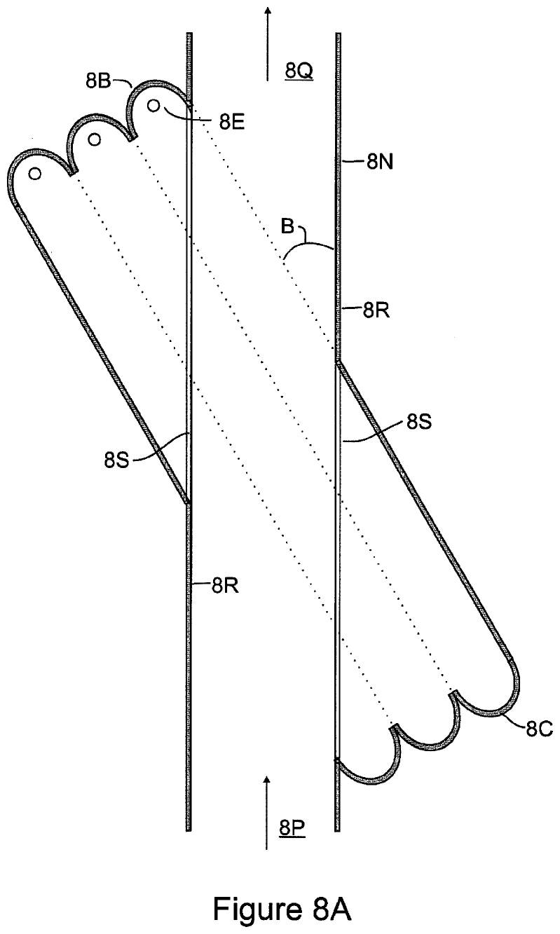

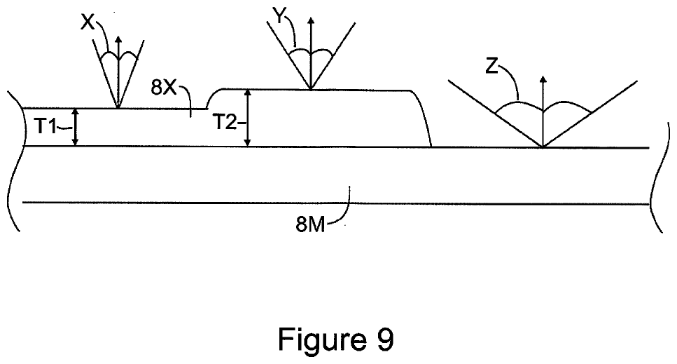

FIG. 8 is a schematic cross-sectional view of a method for directing electromagnetic radiation into to reactive materials in a reaction chamber. In this embodiment the chamber is for example a duct where fluid is constrained to pass and the electromagnetic radiation can be UVC light typically at wavelengths between 220 nm and 280 nm arranged to sterilize materials in the fluid.

Arrangements of this type are described in detail in the above cited MPS application so that reference may be made to this further detail.

As described in the above application, the probability of the electromagnetic radiation interacting with the reactant materials is increased by using multiple reflections from highly reflective surfaces to increase the optical path length of the electromagnetic radiation through the reaction chamber for which the amplitude of said electromagnetic radiation exceeds a threshold value. The energy density within a volume element of the reaction chamber is obtained by summing the amplitudes of radiation paths that pass through the volume element weighted by each path length in the volume element. The threshold value is selected such that the sum of amplitudes below the threshold does not alter the energy density sum by more than a tolerance value. Empirically a threshold value of 0.01% of the initial electromagnetic radiation amplitude was found to work well. The probability of interaction is proportional to the energy density in each volume element and hence correlated with the path length of the electromagnetic radiation. Also as described, the reaction chamber 8A includes a pair of opposed reflective surfaces 8B and 8C of the chamber where at least one, and typically both of the reflective surfaces of each pair is a concave mirror. The surfaces 8B and 8C cooperate with a source 8D or 8E of the radiation which is arranged relative to the surfaces to cause the reflective surfaces of each pair to generate reflections of the electromagnetic radiation back and forth between the reflective surfaces within a volume defined by the reflective surfaces. The surfaces 8B and 8C are made smooth and highly reflective so that the amplitude of electromagnetic radiation reflected back and forth between the surfaces remains above threshold amplitude for at least ten and preferably more than one hundred specular reflections. The path length that the electromagnetic radiation contributes to the energy density between the surfaces becomes approximately the distance between surfaces 8B and 8C multiplied by the number of reflections. The source can be located as indicated at 8D on or adjacent one of the surfaces (8B or 8C) or can be located as indicated at 8E at the focal point of the surface.

The reflective surfaces 8B and 8C spaced one from the other so as to define a first side 8F of the volume on one side of the reflective surfaces and so as to define a second side 8G of the volume on an opposed side of the reflective surfaces.

In this embodiment as shown in FIG. 8 the complete chamber 8A is defined by a plurality of these chambers 8H, 8I, 8J, 8K defined by the pairs which are stacked so as to be arranged side by side. In this way the side 8G of the chamber 8H is coincident with the side of the next adjacent chamber 8I and is open therebetween so that radiation escaping through the side 8G of one volume 8H enters a side of the next adjacent volume 8I. This arrangement is continued through the stack so that each sub-chamber connects to the next at the open sides. The two ends are closed by reflective closure walls 8L and 8M to form the stack into a closed duct defined by the sides 8L and 8M and by the stack of curved walls 8C, 8D.

The plurality of pairs forming the sub-chambers thus define a stack of the volumes defined by the sub-chambers side by side where the radiation can pass between each volume and the next adjacent volume.

It has been found that the ability of the radiation to pass into the next volume or sub-chamber allows that radiation to continue to be reflected in that next volume rather than to be potentially lost. Most loses have been found to occur at the sides of the volumes so that the recapture of these radiation loses significantly increases the overall efficiency and the number of reflections obtained. It will be appreciated that an increase in reflections in each beam increases the magnification effect described in the above cited MPS application.

In FIG. 8 the flow is at right angles to the sides and typically is directed generally longitudinally of the duct.

FIG. 8A is an alternative schematic cross-sectional view similar to that of FIG. 8 of an alternative arrangement for directing radiation into a chamber for fluid flow. In this embodiment is shown a duct 8N with an inlet end 8P and an outlet end 8Q where the radiation is directed at an angle B to a longitudinal direction of the duct. The duct is formed with reflective walls 8R and with transparent sections 8S so that the radiation passes through a window in side walls of the duct. In this way the concave reflective mirrors 8B and 8C and the sources 8E are located outside the duct as a separate element allowing a retrofit to existing ducts. The number of sub-chambers so formed can of course vary in accordance with geometry of the system. The sides of the chamber formed by the end sub-chambers of the stack are not in this arrangement closed by walls.

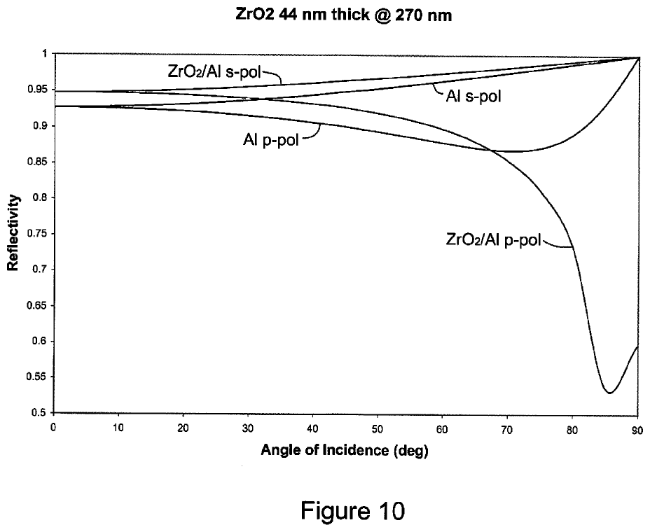

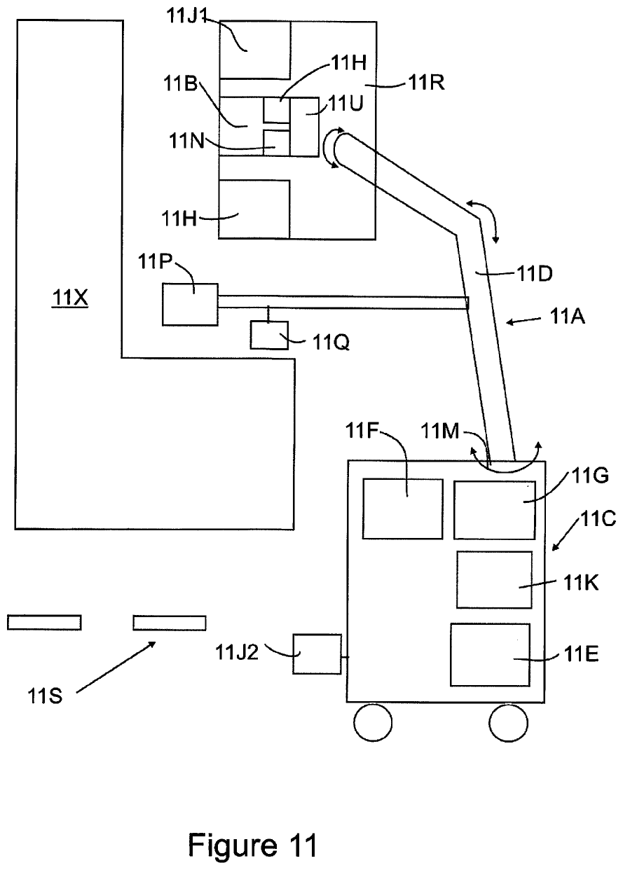

Turning now to FIGS. 9 and 10, FIG. 9 is a cross-sectional view through a portion of the wall of the chamber of FIG. 8 or FIG. 8A showing that the wall is coated with a layer of ZrO₂ with thickness chosen to produce constructive interference at a predetermined design wavelength and hence high reflectivity. FIG. 10 is a graph showing the effect on reflectivity of the arrangement of FIG. 9 where the wall is coated with a reflective layer of ZrO₂.

Thus in this embodiment there is provided a method where at least one reflective surface 8M of the reaction chamber 8A is formed by a metallic reflective wall, typically aluminum, at least part of which is coated with a layer 8X of ZrO₂ (Zirconium dioxide) or HfO₂ (Hafnium dioxide). These materials have a high refractive index and low absorption of radiation for predetermined wavelengths in the UVC range.

The layer 8X is applied with a thickness T1 and T2 selected to increase the reflectivity (by constructive interference) of the radiation at the reflective surface to a value greater than that of the metallic layer alone. As shown, the thickness T1, T2 is varied at different locations on the surface and particularly the thickness is varied at different locations on the surface in dependence on an angle X or Y of incidence of the radiation on the surface so that the thickness is adjusted to optimize reflectivity via constructive interference at each angle of incidence. The constructive interference condition may be met at increasing angles of incidence by decreasing the layer thickness giving increased reflectivity over a broad range of wavelengths. Preferably the layer thickness is increased at locations of greater angle of incidence, which gives a higher reflectivity maximum over a narrower range of wavelengths centered on a design wavelength. The thickness can be varied in steps as shown but more preferably is gradated depending on the angle of incidence of the radiation expected or calculated to impinge on the location concerned. In the preferred embodiment, the thickness is increased depending on the angle of incidence up to a maximum which can be practically obtained. Thus as shown in FIG. 10, an initial thickness of 44 nm provides a level of reflectivity for radiation with a wavelength of 270 nm which is increased relative to that of bare aluminum so as to reduce reflection losses and thus increase the number of reflections that occur, as explained in the above cited MPS application. It will be noted from the graph that the level of reflectivity reduces for p-polarized radiation as the angle of incidence increases so that it is necessary to increase the thickness at these angles up to at least 50 nm and preferably as much as 64 nm. Specifically, the ZrO₂ layer thickness is increased with increasing angle such that radiation reflected from the ZrO₂ surface interferes constructively with radiation reflected from the aluminum surface at the specified angle of incidence.

However in some cases the maximum thickness that can be achieved in a practical application method is limited so that in that situation the material is omitted so that the metallic wall is bare at locations of angle of incidence greater than a predetermined value. Thus for example a layer of ZrO₂ may be added to an aluminum surface by reacting ZrF₆ with the aluminum surface in the presence of a small amount of water. This process is self-limiting to a thickness of about 50 nm. In this case it is preferable to leave regions of the aluminum surface bare where the average angle of incidence requires a coating thickness greater than 50 nm.

The coating of the layer also has the advantage that the material provides an increased hardness relative to the metallic wall thus reducing marring by scratches which would reduce specular reflectivity.

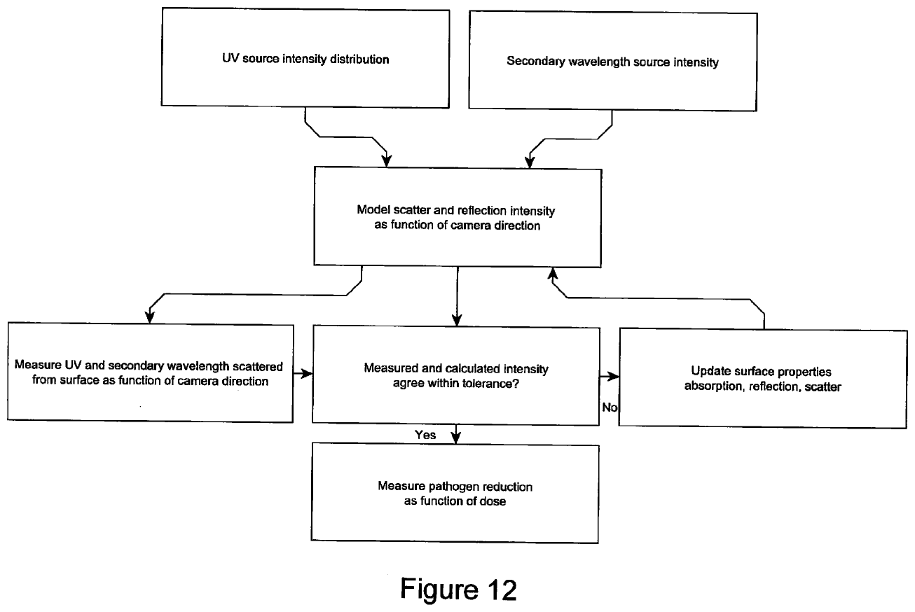

Turning now to FIGS. 11, 12 and 13 there is shown an arrangement using the directed decontamination beam of FIG. 3 to effect decontamination of a body such as a vehicle seat between uses by different passengers.

The vehicle decontamination system 11A shown in FIG. 11 includes a directional UV radiation source 11B, provided for example by the construction as shown in FIG. 3 as described above, a control unit 11C, and a positioning actuator 11D.

The control unit 11C includes a processor 11E together with data storage 11F and communication interface 11G. The control unit data storage includes a dose map in the form of a three-dimensional model of the surface locations to be decontaminated, for example passenger seat 11X, and a set of properties associated with each location. The properties stored may include the surface normal, the reflectivity, scatter and absorbency as functions of the angle of incidence, and information about dose sensitivity. For example, a smooth metal surface may require a lesser decontamination dose than a rough cloth surface. The surface properties may include historical information related to probability of contamination. For example, if a person known to be infected with a pathogen was at the location concerned, the probability of contamination at that location and proximate locations is higher than the average probability of contamination.

The control unit 11C may be linked with sensors 11H at the source 11B that measure the temperature, pressure, humidity and molecular composition of the fluid (in most cases air) between a surface location and the directional UV source 11B.

The surface properties associated in the data storage 11E with each location further include the dose of UV radiation required to achieve a given level of pathogen reduction for each type of pathogen known or expected to be present. For example, the dose required to achieve a log 3 reduction for a given pathogen population may be different depending upon whether the pathogen is on a metal or cloth surface. The dose requirement stored for each surface type is preferably previously measured directly with calibration samples. Specifically, a plurality of samples of each surface type is inoculated with known concentrations of pathogen and then each sample is subjected to different sets of UV dose. The log reduction is then determined by measuring the number of pathogens viable relative to the initial number.

The location properties may further include the risk or probability that contamination at that location has been transmitted to a second surface at the location by contact. For example the second surface may be one contacted by a human hand and the transmission probability will depend upon the surface material and the probable contact time. For example a touch screen may have a high transmission probability and a ceiling that is rarely contacted may have a low transmission probability. It is worth noting that small particles are constantly adsorbing and desorbing from surfaces with a temperature dependent residence time. Hence pathogen particles on a first surface with low transmission probability may migrate to a surface with higher transmission probability. The data storage 11E preferably takes into account the time dependent probability of migration in a risk weighted model.

The control system 11C can receive at the interface 11F a pathogen reduction target from a user and calculates from that target the risk weighted dose required at each surface location based on the surface properties, transmission probability and dose sensitivity to meet that target pathogen reduction. The positioning actuator 11D then positions and orients the directional UV radiation source 11B to deliver the required dose to each surface location.

The positioning movement of the source may be provided by a human operator moving and orienting the decontamination system in response to instructions and feedback from the control system 11C. In this embodiment the control system 11C may use sensors 11J1 and 11J2 to determine the position and orientation of the decontamination system (and hence the position and orientation of the directional UV source) and calculate the dose delivered to each location to be decontaminated based on said directional UV source location and orientation. The control system 11C may generate visual and acoustic signals to the human operator with information about which surfaces have received a sufficient dose and which surfaces have not received a sufficient dose.

In a preferred embodiment the positioning actuator 11D is a robot that guides the decontamination system along a controlled path. In this embodiment, the control system 11C further includes subsystem 11K that measures the position and orientation of the decontamination system and subsystems 11M that operates to position the decontamination system by driving motors controlling the actuator 11D. Preferably the positioning subsystem is operable to position and orient the directional UV source with six degrees of freedom (arbitrary position and orientation). In some embodiments, fewer number of degrees of freedom may be used. The control system 11C may calculate a plurality of decontamination system paths that meet the user supplied decontamination target. The control system 11C then selects a path from the plurality of paths that meet the decontamination target. The path selected may for example be a path that minimizes the time required for decontamination. Alternately the path selection algorithm may minimize the energy required for decontamination. The control system 11C then generates signals to actuator 11D that cause the decontamination system to move along the selected path.

Optionally, the decontamination system includes a detector 11N that measures the UV source intensity and the control system 11C uses the measured source intensity to dynamically adjust the exposure time at each location based on the measured source intensity such that a required dose is delivered to each location. This feature is useful to compensate for the decline in radiation source intensity as the radiation source ages. Further, if the measured source intensity falls below a threshold value, control 11C may generate a signal to a operator that maintenance (source replacement) is required.

Optionally, the decontamination system includes a detector defined by a camera 11H that measures radiation reflected or scattered from a surface location and the control system 11C uses the intensity received at the detector 11N together with surface properties of the location to calculate the dose received at the surface location and adjusts the exposure time such that the required dose is delivered to each location. As discussed in more detail below, the wavelength(s) measured by the camera 11H may be different from the wavelength(s) of the collimated beam generated at 11B used for decontamination. For example, the camera may measure the intensity of visual wavelengths (400-800 nm) and the decontamination wavelengths may be between 220 nm and 280 nm.

Optionally, the decontamination further includes a probe 11P to collect samples from surface locations before or after irradiation and the collected samples collected are analyzed for viable pathogens. This feature may be used for example to determine whether the dose is sufficient for the pathogens actually encountered as opposed to pathogens expected. Note that the pathogen types may change due to mutations or the emergence of new types. The analysis may be conducted by standard wet chemical methods. Preferably the analysis is done using rapid methods described by the above cited HEMS patent.

Optionally the decontamination system further includes a collection means 11Q operable to collect particles from the surface of object 11X. Collection means 11Q may include an agitator and a collector. The agitator may be mechanical or a stream of pressurized gas and the collector is an aspirator. The agitator operates on a surface location to dislodge adsorbed particles (including pathogen particles) and the aspirator draws the particles so dislodged into a stream for treatment or measurement. For treatment, the stream of dislodged particles may be directed into a Multipass Photochemistry chamber as disclosed in the MPS application cited above.

In some embodiments agitator and collector 11Q are the arrangement described in Multiple Pass Imaging Spectroscopy U.S. Pat. No. 8,345,254 issued Jan. 1 2013 to Prystupa, the disclosure of which is incorporated herein by reference or which may be referenced for further detail.

Sample material dislodged from the surface of object 11X by agitator and collector 11Q may be processed and examined for the type and number of micro-organisms present by various methods described below. The agitator and collector 11Q may be used to randomly sample locations on the surface of object 11X and analysis of particles collected at sample locations provides detailed information about materials and contaminants present at that location. The spatial distribution of materials and contaminants may be analyzed by control 11C to build statistical models of contamination probability with location and to detect systemic problems with sanitization procedures in a manner analogous to the way food products are statistically sampled to detect sanitation problems in processing protocols and equipment. The information from randomly sampled locations on object 11X may be used to build statistical models that predict the probability of contamination as a function of location. As noted above, the location dependent probability may be used to optimize allocation of UVC dose: that is to allocate a finite dose among different locations so as to minimize either the number of pathogens remaining overall or to minimize the probability of pathogen transmission to a human using location weighted transmission probabilities. For example, a human is more likely to interact with a touch screen than a ceiling, so a higher log reduction of potential pathogens on the touch screen than the ceiling will reduce the probability of transmission to a human to a greater extent than if the touch screen and ceiling were treated with beam produced by collimated source 11B to give equal log reductions of potential pathogens. In some embodiments samples are collected by collector 11Q from a location prior to sanitization by UVC irradiation by directional source 11B and control 11C determines the directional UVC dose delivered to said location at least in part based on measurements of a sample from said location. For example, control 11C may, based on risk, allocate a higher dose of UVC radiation to a first location with a higher than average measured contamination level (or transmission probability) and a lower dose of UVC radiation to a second location with a lower than average measured contamination level (or transmission probability). For example, control 11C may infer the most probable spatial distribution of contamination on object 11X from a limited set of random locations using the methods of compressive imaging known to those skilled in the art. Control 11C may further determine a risk weighted dose of UVC irradiation from directional source 11B for locations not directly sampled based at least in part from the most probable spatial distribution of contamination.

In some embodiments probe 11P and/or agitator and collector 11Q are used to determine the presence of viable micro-organisms at a location following irradiation by directional UVC source 11B. In this embodiment the information may be used to validate the sanitization process and to document the efficacy of the sanitization process.

Biological samples from agitator and collector 11Q may for example be transported to and deposited onto appropriate optical substrates using a micro-fluidic system. Preferably the micro-fluidic system is the arrangement described in published PCT application WO 2021/163799 published Aug. 26 2021 by the present inventors entitled Field Programmable Fluid Array the disclosure of which is incorporated herein by reference or which may be referenced for further detail.

In some embodiments surface enhanced Raman and infrared spectra may be collected by placing biological samples from collector 11Q onto magnetic objects as described in published PCT application WO 2021/163798 published Aug. 26 2021 by the present inventors entitled Magnetic Platform for Sample Orientation.

Preferably the spectra are measured using the arrangement described in the above cited HEMS patent, which provides a superior signal-to-noise ratio the disclosure of which is incorporated herein by reference or which may be referenced for further detail.

In some embodiments biological sample material is placed in the arrangement described in the above cited Multiple Pass Imaging Spectroscopy patent and optical amplification is used to increase the signal level and reduce the measurement time. In other embodiments, the surface is sampled directly by probe 11P using the internal reflectance arrangement described in the above cited Multiple Pass Imaging Spectroscopy patent. Preferably the amplified absorption spectra are measured with the above cited HEMS method. In some embodiments biological material from collector 11Q is placed in the arrangement described in U.S. provisional patent application 63/120,318 entitled Amplified Multiplex Absorption Spectroscopy filed Dec. 2, 2020 by the present inventors. Preferably the amplified absorption spectra are measured with the above cited HEMS method. In some embodiments biological material from collector 11Q is placed in the arrangement described in U.S. patent application Ser. No. 17/387,553 entitled Multi-dimensional Spectroscopy filed Jul. 28, 2021 by the present inventors, now published on Feb. 3 2022 as 2022/0034817. Preferably the multi-dimensional spectra are measured with the above cited HEMS method. In some embodiments the biological material from collector 11Q is tested for biochemical composition, for example DNA or RNA. In this embodiment the speed of the test may be increased using the arrangement described in U.S. patent application Ser. No. 17/387,533 entitled Directed Orientation Chemical Kinetics filed Jul. 28, 2021 by the present inventors now published as PCT WO 2022/020955. The data from the above cited spectral and chemical methods is preferably analyzed to determine the types of micro-organisms present by using the methods described in U.S. provisional patent application 17/535,034 entitled Spectral Diagnostic System filed Nov. 24, 2021 by the present inventors and now published as US 2022/0170839 on Jun. 16 2022 the disclosure of which is incorporated herein by reference or which may be referenced for further detail.

In some embodiments, detector 11H is a multi-spectral imaging camera. Preferably the multi-spectral imaging system is the arrangement described in the above cited HEMS patent by the current inventors. Other multi-spectral imaging systems may be used. The multi-spectral imaging system provides images of object 11X, or parts thereof, for at least three different wavelengths, more preferably more than 100 different wavelengths and most preferably more than 1000 different wavelengths. In this embodiment, the entire surface of object 11X may be scanned and locations requiring sanitation are determined at least in part based on the spectral profile of each location. The spectrum of each location is found by mapping each location to a region of the spectral image by methods known in the art (comparing measurements from image and distance sensors with a three-dimensional model of the environment). That is the spectrum of each location is compared with spectra in a spectral database and the composition of material at each location is determined at least in part by matching the location spectrum with a combination of one or more known reference spectra by control 11C. The spatial resolution of the multi-spectral imaging system 11H is selected to resolve the smallest contamination particle known or expected to be present. For example, the inventors determined that spatial resolution of approximately 0.3 mm is required to detect the presence of fecal contamination on surfaces. Control 11C determines the UVC dose for each location on object 11X based at least in part on the materials determined to be present at the location by analysis of the spectrum from the location.

The method herein thus includes collecting a multi-spectral image of at least a portion of the body; determining at least in part the type of contamination at different locations within said multi-spectral image by comparing the spectrum of each location with reference spectra; and directing the beam to a location within said multi-spectral image based at least in part on the type of contamination. Preferably each spectrum within the multi-spectral image is comprised of more than three different wavelengths. However, three or less can also be used. Preferably each spectrum within the multi-spectral image is comprised of more than one hundred different wavelengths.

Optionally the decontamination system includes a position verification system 11J1 as a component of the head 11R mounting the source 11B, which may be a camera operating together with software to track as a function of time the locations the directional UV source is pointed toward. The position verification means may include a LIDAR unit which measures the distance from the directional UV source to a surface location. The position verification system may include an acoustic unit which measures the distance from the directional UV source to a surface location. The control system 11C may use the time, distance and location information, together with calibration information about the spatial distribution of radiation from the radiation source to calculate the dose received (or to be delivered) at each location.

In some embodiments the dose information is displayed on the interface 11F on an overlay image to an operator wherein the overlay image contains an image of the surface to be decontaminated together with suitable color representations of the dose received at each location relative to the dose required at each location. The interface 11F may for example be a touch screen or a cell phone screen wherein a data link is provided between control 11C and the display means. For example, locations that have received zero dose may be shaded red, locations that have received an incomplete dose may be shaded yellow, locations that have received the desired dose may be shaded green, and locations that have received an excess dose may be shaded blue. Other shading schemes may be used and the number of shades may be varied to suit the sophistication of the operator. In preferred embodiments the position and dose information is stored in a database. The database information may be used to confirm that the above user specified decontamination target is met. The database information may be used in combination with pathogen indicators to adjust the dose.

Optionally the camera 11H of the decontamination system may act for dose monitoring that measures radiation reflected and scattered from a location to calculate the intensity of radiation received at the location. The reflected and scattered radiation may be the primary decontamination radiation at source 11B at a UV wavelength or a secondary wavelength of radiation from a source 11U mixed with the primary radiation in a fixed proportion. In some embodiments a detector such as the camera 11H sensitive to the primary UV wavelength measures the intensity of UV radiation reflected and scattered from a location. The control system 11C uses the intensity information together with the bidirectional reflectance function (BDRF) of the location (previously measured) to calculate the intensity received at the location. For example, the previously measured BDRF may indicate that 1% of radiation received from the source direction is reflected in the direction of the detector 11H. In this case the intensity at the location is calculated as 100× the intensity received at the detector. In some embodiments, the second wavelength from source 11U is mixed in fixed proportion to the primary wavelength in the directional UV source and the second wavelength is measured by a detector such as the camera 11H. The second wavelength preferably has similar reflectivity and scattering characteristics to the primary UV wavelength. The secondary wavelength may for example be a blue wavelength between 405 nm and 480 nm that is easily measured with a silicon-based photodiode or photodiode array.

The number N of pathogens transferred to a host species such as a human from object 11X may be calculated as

$$N = \sum {A}_{i} {P}_{i} exp \{ - {k}_{i} {f}_{i} {t}_{i} \}$$

Where Aᵢ is the area of the ith region of object 11X; Pᵢ is the probability transmission from the ith region to a host species; kᵢ is the effective decay constant for the pathogen at the ith region; fᵢ is the radiation flux at the ith region; and tᵢ is the time the flux is directed at the ith region. The sum is over all values of i. The probability of transmission Pᵢ from each region may be measured experimentally or inferred from statistical analysis of known transmission cases. The probability Pᵢ will in general vary between different surface materials due to differences in binding energy between the surface and pathogen and due to differences in the path from a pathogen residence site to an external host species. For example, a pathogen embedded in a fabric may be required to transit multiple binding sites to reach a host species whereas a pathogen on a smooth surface may be transferred directly to a host species by overcoming only one surface binding energy.

The effective decay constant kᵢ is based on decay constant for the pathogen species k modified by the environment at region i. The decay constant k is measured experimentally for standard conditions and is reported in the scientific literature for hundreds of pathogen species. The environmental modification from standard conditions may be due to geometric shading effects or due to differences in temperature and humidity. For example, fibers in a fabric surface may absorb radiation and reduce the effective radiation dose at the pathogen. As noted above, the temperature and humidity may be measured and used to calculate an environmentally modified decay constant.

One advantage of the present invention is that the flux factor fᵢ has minimal spatial variation due to beam collimation and can be approximated as a constant. The dose for each region is then proportional to the time tᵢ the beam is directed at the region.

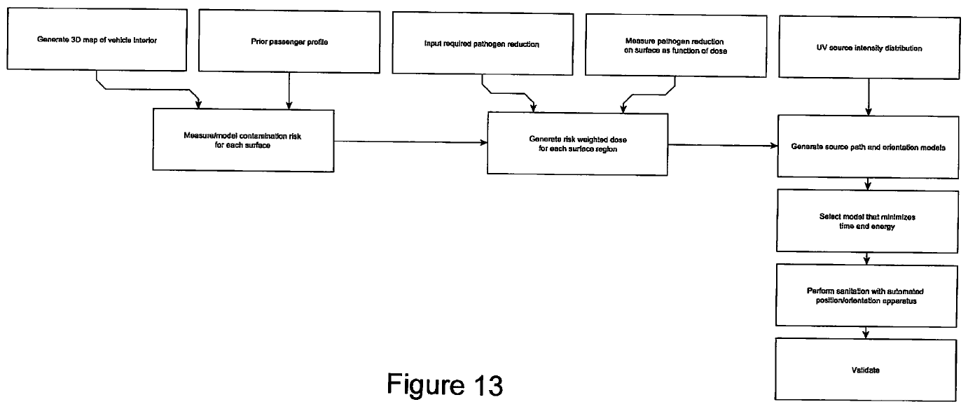

FIGS. 12 and 13 comprise flowcharts setting out the operations described above as carried out by the control system 11C.

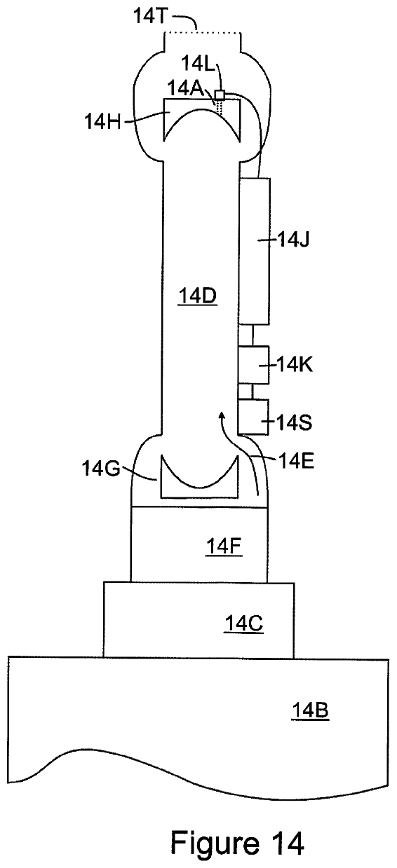

FIG. 14 schematically shows an arrangement for sterilizing a flow of water generally indicated as the area within dashed box 14A. The arrangement uses elements of the reaction chamber for sterilization described in the above cited MPS patent by the present inventors. A supply of water is contained in a vessel 14B. The vessel 14B may for example be a water bottle or storage container of conventional design. The vessel 14B may for example be a water pitcher. The vessel 14B may for example be a water pipe or a water faucet. Vessel 14B may include an integral attachment means that may for example be a threaded section of conduit. In other embodiments sterilizing attachment 14D is a conduit that attaches to vessel 14B by a press fit. In other embodiments sterilizing arrangement 14D may be suspended in vessel 14B or attached to a side of vessel 14B with a clip. The arrangement 14D may be attached to water vessel 14B via a coupler 14C. Coupler 14C may for example be a fitting with female threads designed and fabricated to match male threads on the integral attachment means of water vessel 14B. Coupler May be different for each type of water vessel 14B, but provides a common interface to the remaining components of water sterilization apparatus 14D. That is the apparatus 14D may be adapted to any conventional water vessel by selecting an appropriate coupler 14C. Coupler 14C is in communication with water filter 14F which functions to reduce particulate matter and optionally reduce selected chemical contaminants. Water flows from water vessel 14B through coupler 14C and filter 14F into sterilization chamber 14D. Sterilization chamber 14D includes opposing high reflectivity concave mirrors 14G and 14H. Water may flow around the edge of concave mirror 14G into chamber 14D as shown at 14E. Similarly, water may flow around the edge of concave mirror 14H toward and through outlet 14T. Hence water may flow continuously from water vessel 14B to water outlet 14T. UVC radiation at wavelengths between 200 nm and 290 nm is admitted to sterilization chamber 14D through aperture 14A in concave mirror 14H from light source 14L. The path length of the UVC radiation above a threshold amplitude in chamber 14D is increased by reflections between concave mirrors 14G and 14H thereby amplifying the sterilizing effect of UVC light admitted to chamber 14D by a factor of at least 10 and preferably by a factor of 100 or more. The amplification is achieved by using highly reflective dielectric mirrors at 14G and 14H and by selecting suitable chamber geometry as described in more detail in the above cited MPS patent by the current inventors. Light source 14L may for example be a LED that is connected to a supply of electrical power 14J under the control of control means 14K. Alternately, light source 14L may for example be connected by a waveguide to a light source as shown in FIG. 2, 3 or 4B. Control means is in communication with sensor 14S that is operable to measure at least one property of the water and optionally a plurality of properties. The properties may for example be flow rate, temperature, turbidity, conductivity and pH. In one embodiment, control means may include a user interface that allows a user to manually switch the sterilization function on, control the amplitude of UVC radiation, and provide functional status information. In another embodiment the control means may activate the UVC light source automatically when water flow is detected.

1. A method for applying electromagnetic radiation to reactive materials in a reaction chamber comprising:
introducing the electromagnetic radiation into the chamber;
and increasing the probability of interaction of the electromagnetic radiation with the reactant materials by using multiple reflections to increase the optical path length of the electromagnetic radiation within the reaction chamber for which the amplitude of the electromagnetic radiation is above a threshold value;
wherein the reaction chamber includes a plurality of pairs of opposed reflective surfaces of the chamber;
wherein at least 50% and more preferably at least 80% or 90% of the reflections from the reflective surfaces are specular reflections;
wherein at least one of the reflective surfaces of each pair is a concave mirror;
the reflective surfaces of each pair being arranged to cause reflections of the electromagnetic radiation back and forth between the reflective surfaces within a volume defined by the reflective surfaces;
the reflective surfaces of each pair being spaced one from the other so as to define a first side of the volume on one side of the reflective surfaces and so as to define a second side of the volume on an opposed side of the reflective surfaces;
wherein the pairs are arranged side by side so that radiation escaping through a side of one volume enters a side of a next adjacent volume.

2. The method according to claim 1 wherein the plurality of pairs define a stack of the volumes side by side where the radiation can pass between each volume and a next adjacent volume.

3. The method according to claim 2 wherein end ones of the volumes have a reflective side wall on an outer one of the sides thereof.

4. The method according to claim 1 wherein the reflective surfaces form side walls of a duct.

5. The method according to claim 1 wherein the flow is at right angles to the sides.

6. The method according to claim 1 wherein the radiation is directed into a duct through which a fluid passes.

7. The method according to claim 6 wherein the radiation is directed generally longitudinally of the duct.

8. The method according to claim 6 wherein the radiation is directed at an angle to a longitudinal direction of the duct with the radiation passing through a window in side walls of the duct.

9. The method according to claim 1 wherein a majority of radiation paths include at least ten and preferably more than one hundred reflections from surfaces bounding the reaction chamber.

10. The method according to claim 1 wherein the reflective surfaces define at least one center optical axis extending therebetween along which the reflections pass and wherein a source of the radiation is located at a position offset from the center axis between the reflective surfaces so that a locus of the reflections moves toward the center axis.

11. The method according to claim 1 wherein a source of the radiation is located at one side of said at least one reflective surface of a reflective pair.

12. The method according to claim 1 wherein the reflective surface is a concave mirror and a source of the radiation source is located at a position on said at least one concave mirror and wherein the source of the radiation has a dimension which is less than 0.03 times the focal length of the mirror.

13. The method according to claim 1 wherein a source of the radiation source is located at a focal point of the concave mirror.

14. The method according to claim 1 wherein the offset between each beam and a next beam after a reflection is less than a width of the beam so that the beams form a complete curtain.

15. The method according to claim 1 wherein there is provided an inlet port for admitting reactive materials and an outlet port for discharging product materials and wherein there is provided absorbing surfaces formed and shaped to stop transmission of electromagnetic radiation from the interior of the chamber to an exterior location.

16. The method according to claim 15 wherein the inlet and outlet ports are not on an axis of symmetry of the reaction chamber.

17. The method according to claim 1 wherein at least part of a chamber wall reflects electromagnetic radiation diffusely.

18. The method according to claim 1 wherein the reactive material is entrained in a fluid flow wherein the fluid is a liquid or a gas.

19. The method according to claim 1 wherein the electromagnetic radiation is UVC radiation and the reactive material is a microorganism selected from the list of bacteria, virus, protozoan, helminth, yeast, mold or fungus and said UVC radiation inactivates said microorganism.

20. The method according to claim 1 wherein the electromagnetic radiation is at least partially collimated to travel primarily back and forth between the reflective surfaces.

21-160. (canceled)
